(19) United States
(12) Patent Application Publication
(10) Pub. No.: US 2024/0139278 A1
(43) Pub. Date: May 2, 2024

(54) OXYTOCIN FORMULATION

(71) Applicant: XELLIA PHARMACEUTICALS APS, KØBENHAVN S (DK)

(72) Inventors: Ivona Jasprica, Zagreb (HR); Marcel Lagedroste, Martinsreid (DE)

(73) Assignee: XELLIA PHARMACEUTICALS APS, KØBENHAVN S (DK)

(21) Appl. No.: 18/392,896

(22) Filed: Dec. 21, 2023

Related U.S. Application Data
(63) Continuation-in-part of application No. PCT/EP2023/067134, filed on Jun. 23, 2023.
(60) Provisional application No. 63/489,486, filed on Mar. 10, 2023.
(60) Provisional application No. 63/355,195, filed on Jun. 24, 2022.

(51) Int. Cl. A61K 38/095; A61K 47/12; A61K 47/18; A61K 47/26

(52) U.S. Cl. CPC A61K 38/095; A61K 47/12; A61K 47/183; A61K 47/26

(57) ABSTRACT

The present disclosure relates to a ready-to-administer formulation of oxytocin, its pharmaceutically acceptable salts, or derivatives thereof, as well as products including the formulation. Specifically, the disclosure is directed to storage stable ready-to-administer formulations of oxytocin, method of producing such formulations and uses thereof.

### CROSS-REFERENCE TO RELATED APPLICATIONS

This application is a bypass continuation-in-part of International Application Serial No. PCT/EP2023/067134, filed on Jun. 23, 2023, which claims priority to U.S. Provisional Application Ser. No. 63/355,195 filed on Jun. 24, 2022 and 63/489,486, filed on Mar. 10, 2023, which are incorporated herein by reference in their entirety.

### FIELD OF THE INVENTION

The present disclosure is directed to a ready-to-administer oxytocin formulation and uses thereof. Specifically, the disclosure is generally directed to storage stable formulations of oxytocin.

### BACKGROUND

Oxytocin is a cyclic nonapeptide hormone having a structural formula as follows:

Cys-Tyr-Ile-Gln-Asn-Cys-Pro-Leu-Gly-NH₂, or CYIQNCPLG-NH₂.

Oxytocin is prepared synthetically to avoid possible contamination with vasopressin and other small polypeptides with biological activity.

Oxytocin injection is indicated for the induction of labor in patients with a medical indication for the initiation of labor, such as patients with Rh problems, maternal diabetes, preeclampsia at or near term, when delivery is in the best interests of mother and fetus, or when membranes are prematurely ruptured and delivery is indicated; for the stimulation or reinforcement of labor, as in selected cases of uterine inertia; as adjunctive therapy in the management of incomplete or inevitable abortion; to produce uterine contractions during the third stage of labor; and to control postpartum bleeding or hemorrhage.

Currently, oxytocin is available in a concentration of 10 United States Pharmacopoeia (USP) Units/ml as a single-use vial. The formulation is typically stored in a 1 ml glass vial. The available formulation contains chlorobutanol at 0.5 percent (5 mg/ml) as a preservative and has a pH of 3.0 to 5.0.

The currently approved method of administration of oxytocin for the induction or stimulation of labor is through intravenous (IV) infusion. To prepare the usual solution for IV infusion, 1-mL Oxytocin Injection, 10 USP Units/mL product needs to be diluted to typically 10 mU/mL prior to administration to a patient.

Thus, one of the drawbacks of the existing oxytocin formulations is that, in case of IV infusion, a dilution step is required prior to administration to a patient, thus increasing the risk of medication errors. Further, after initial closure puncture and subsequent dilution, such diluted formulation may be used only for a short period of time and any unused portion should be discarded.

Another drawback of the existing formulation is that it uses chlorobutanol as a preservative. However, chlorobutanol is a skin irritant and is also highly toxic to the liver. Thus, it would be desirable to avoid chlorobutanol in oxytocin formulations.

Thus, there is a need in the art for a ready-to-administer oxytocin formulation which would be stable for certain prolonged periods of time under storage conditions and that could be delivered to a patient without a need for dilution.

Furthermore, there is a need for a stable ready-to-administer oxytocin formulation free of chlorobutanol.

### SUMMARY

The present disclosure relates to a ready-to-administer formulation of oxytocin, its pharmaceutically acceptable salts, or derivatives thereof, as well as products comprising the formulation.

According to the present disclosure, degradation of oxytocin is retarded, and the shelf life of the stable formulation is prolonged.

In one aspect, pH of the formulation is from 3.2 to 5.5.

In one aspect, ready-to-administer formulation is in a unit dosage form.

In one aspect, the concentration of oxytocin in the formulation is from 5 mU/ml to 100 mU/ml.

In one aspect, the pharmaceutical formulation of the present disclosure is in aqueous form.

The inventors have unexpectedly found that when oxytocin is formulated in formulations according to the present disclosure, degradation products formation is retarded, and accordingly, such formulations exhibit prolonged stability and provide more flexible storage conditions and handling when stored under controlled room temperature conditions.

This disclosure also provides a process for manufacturing disclosed pharmaceutical formulations.

The pharmaceutical formulations according to the present disclosure may be used to initiate or improve uterine contractions to a subject in need.

It is to be understood that both the foregoing description and the following further description are exemplary and explanatory only and are not restrictive of the claims.

### DETAILED DESCRIPTION

The present disclosure relates to ready-to-administer oxytocin formulations and products comprising the formulations.

According to the present disclosure, degradation of oxytocin may be retarded and the shelf life of a such product comprising the formulations may be prolonged.

In accordance with the present description, oxytocin is synthetically produced.

In one aspect, the pH of the formulation is from 3.2 to 5.5.

In one aspect, the pH of the formulation is from 3.4 to 5.0.

In one aspect, the pH of the formulation is from 3.6 to 4.5.

In one aspect, the pH of the formulation is from 3.8 to 4.5.

In one aspect, the pH of the formulation is 3.4, 3.5, 3.6, 3.7, 3.8, 3.9, 4.0, 4.1, 4.2, 4.3, 4.4, 4.5, 4.6, 4.7, 4.8, 4.9 or 5.0.

“pH” is the conventional measurement unit of hydrogen ion activity in a solution at controlled room temperature, unless another temperature is specified.

As used herein, the term “pH” in compositions is defined as ±0.1 of the numerical value or range in question.

In one aspect, composition is in unit dosage form.

It was found that when oxytocin is formulated in formulations according to the present disclosure, degradation products formation is retarded, and accordingly, such formulations exhibit prolonged chemical stability and provide more flexible storage conditions and handling when stored under controlled room temperature conditions.

By the term “controlled room temperature” used herein, it is meant a controlled room temperature as set in USP <659>, i.e., from 20 to 25° C.

Further, the formulation in accordance with the present disclosure has improved stability at a temperature of 40° C.

The term “stable” means that the compositions meet one or more of the following criteria:
(i) The composition exhibits an acceptable amount of active ingredient degraded after a certain time period compared to the amount of oxytocin present at the beginning of the time period; and/or
(ii) the composition exhibits an acceptable amount of impurities being formed after a certain time period compared to the amount of impurities present at the beginning of the time period; and/or
(iii) the composition retains a desirable appearance such as clarity, improved color, and no visible particles (i.e., precipitate free or particle free composition) over a period of time. Visual inspection for visible particles may be performed as follows: the container under inspection is gently swirled and inverted, ensuring that no air bubbles are produced, and inspected during certain period (approximately 5 to 20 sec) with the naked eye and/or under a magnifier. Visual inspection for a change of color may be performed as follows: The container is inspected by eye and a color is assigned to the composition. The color may also be determined by a UV/VIS spectrometer and the difference in color between two samples may be expressed as delta E.

As used herein, an acceptable amount of active ingredient degraded after a certain time period (i.e., a drop in oxytocin assay) is calculated as a difference in oxytocin assay between assay determined immediately after formulation preparation and assay determined at specific stability testing point e.g., after 1 month, 2 months, 3 months, etc. The oxytocin assay is analyzed, for example, by liquid chromatography, e.g., HPLC, UHPLC, LC/MS.

In an aspect, the oxytocin assay is analyzed by UHPLC. In an aspect, the oxytocin assay is analyzed by UHPLC-FLD, UHPLC-DAD or UHPLC-MS method.

In one aspect, a ready-to-use composition according to the present disclosure is stable under controlled room temperature conditions for a certain time period.

In one aspect, the compositions are stable for at least 14 days, at least 1 month, at least 2 months, at least 3 months, at least 6 months, at least 9 months, or at least 12 months, when stored under controlled room temperature conditions.

In one aspect, the compositions are stable for at least at least 3 months when stored under controlled room temperature conditions.

Formulations according to the present disclosure showed surprising stability for a reasonable period of time, when stored at a temperature of 30° C.

In one aspect, the compositions are stable for at least 14 days, at least 1 month, at least 2 months, at least 3 months, or more when stored at temperature of 30° C.

Formulations according to the present disclosure showed surprising stability for a reasonable period of time, when stored at a temperature of 40° C.

In one aspect, the compositions are stable for at least 14 days, at least 1 month, at least 2 months, at least 3 months, or more when stored at temperature of 40° C.

In one aspect, “stability” may be defined by the amount of total or individual impurities in the formulation after a certain period. Stability may also be defined by an increase in total or individual impurities generated after a suitable period.

The stability may be determined by measuring the amount of individual impurity in the formulation according to the present disclosure after a predetermined time period, preferably expressed as a percentage or area-percentage as analyzed by HPLC or UHPLC. In an aspect, the amount of individual impurity in the formulation is analyzed by UHPLC. In an aspect, the amount of individual impurity in the formulation is expressed as a percentage or area-percentage as analyzed by UHPLC-FLD or UHPLC-DAD method. In an aspect, the amount of individual impurity in the formulation is expressed as a percentage or area-percentage as analyzed by UHPLC-MS method.

By the term “impurity” as used herein is meant a degradation impurity of the active pharmaceutical ingredient in the pharmaceutical formulation.

In an aspect, by the individual impurities, it is meant any larger species that can be generated through covalent or non-covalent interactions of at least two monomers, monomer(s) with degradant(s) of monomer or at least two degradant(s) of monomer. Such species may be dimers, trimers, tetramers or bigger oligomers, and even higher-order structures with higher molecular weight, reversible and non-reversible. Such species may be analyzed using the methods known in the art, such as the one described in the Avanti et al. (“A New Strategy to Stabilize Oxyotcin in Aqueous Solutions: I. The Effects of Divalent Metal Ions and Citrate Buffer”, The AAPS Journal, 13(2), pp. 284-290, 2011) and appropriate modifications thereof to ensure adequate sensitivity.

By the individual impurities related to oxytocin, it is predominantly meant following impurities: oxytocin trisulfide and monodeamidated oxytocin.

The structure of oxytocin trisulfide is shown below as Structure 1.

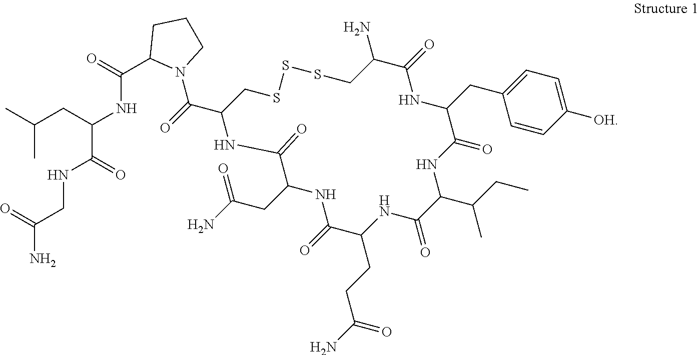 [SMILES: CCC(C)C1NC(=O)C(Cc2ccc(O)cc2)NC(=O)C(N)CSSSCC(C(=O)N2CCCC2C(=O)NC(CC(C)C)C(=O)NCC(N)=O)NC(=O)C(CC(N)=O)NC(=O)C(CCC(N)=O)NC1=O]

The structure of monodeaminated oxytocin is shown below as Structure 2.

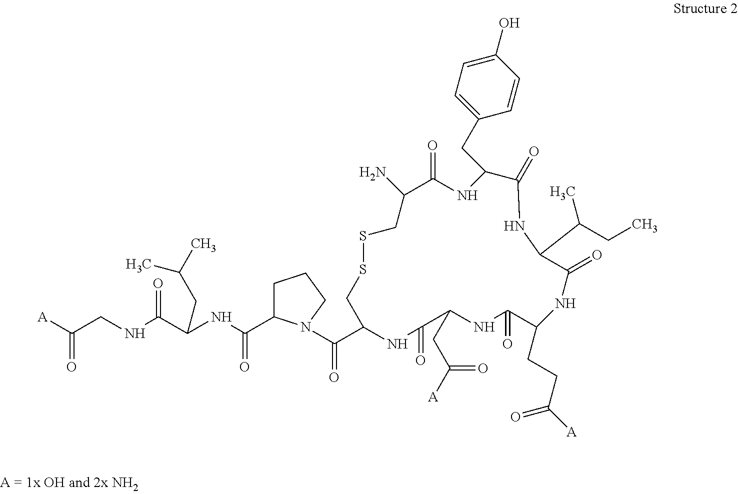

Structure 2. Representative formula of monodeamidated oxytocin (three possible structures), where one of the A is OH group and other two A's are NH2 groups and where OH group and NH2 groups can be situated in all three A positions.

In an aspect, the oxytocin formulation may be stressed at 70° C. for 2 to 5 days. Stress testing or forced degradation is a rapid method of determining the degradation pathways of a drug.

In an aspect, one of the degradation products of oxytocin is intermolecular dimer(s). Without being held to theory, it is believed that formation of dimer(s) is observed in certain oxytocin formulations when they are stressed such as at 70° C. for 2 to 5 days. Unexpectedly, certain oxytocin formulations according to the present disclosure show a reduced or minimal formation of dimer(s) when stressed.

In an aspect, the disclosed formulations also minimize the degradation of the active pharmaceutical ingredient to impurities.

In an aspect, the disclosed formulations retain a pharmaceutically desirable appearance such as no visible particles.

In a specific aspect, “stable” is defined as no more than a 10% drop of oxytocin assay in the pharmaceutical formulation, analyzed by liquid chromatography, e.g., HPLC, UHPLC, LC/MS.

For example, a stable composition can be one which has not more than 1%, 2%, 3%, 4%, 5%, 6%, 7%, 8%, 9%, 10% drop of oxytocin assay after a predetermined time period analyzed by liquid chromatography, e.g., HPLC, UHPLC, LC/MS.

For example, a stable composition can be one which has not more than 1%, 2%, 3%, 4%, 5%, 6%, 7%, 8%, 9%, 10% drop of oxytocin assay after a predetermined time period analyzed by liquid chromatography, e.g., HPLC, UHPLC, LC/MS.

In an aspect, a stable composition can be one which has not more than an 8% drop of oxytocin assay after a predetermined time period.

In an aspect, a stable or stabilized composition can be one which has not more than a 10% drop of oxytocin assay after certain period.

In an aspect, a stable or stabilized composition can be one which has not more than an 8% of drop of oxytocin assay after 1 month at temperature of 40° C., analyzed by liquid chromatography, e.g., HPLC, UHPLC, LC/MS.

In an aspect, a stable or stabilized composition can be one which has not more than a 10% drop of oxytocin assay after 2 months under controlled room temperature as analyzed by UHPLC.

In an aspect, a stable or stabilized composition can be one which has not more than a 10% drop of oxytocin assay after 3 months under controlled room temperature as analyzed by UHPLC.

In an aspect, a stable or stabilized composition can be one which has not more than a 10% drop of oxytocin assay after 4 months under controlled room temperature as analyzed by UHPLC.

In an aspect, a stable or stabilized composition can be one which has not more than an 8% drop of oxytocin assay after 4 months under controlled room temperature as analyzed by UHPLC.

In an aspect, a stable or stabilized composition can be one which has not more than an 8% drop of oxytocin assay after 3 months under controlled room temperature as analyzed by UHPLC.

In an aspect, a stable or stabilized composition can be one which has not more than a 10% of drop of oxytocin assay after 6 months under controlled room temperature as analyzed by UHPLC.

In an aspect, a stable or stabilized composition can be one which has not more than an 8% drop of oxytocin assay after 6 months under controlled room temperature as analyzed by UHPLC.

As used herein, in the case where more than one impurity of the individual active pharmaceutical ingredient can be formed over time, “stable” is defined as no more than a 5% increase of each individual impurity formation after a predetermined time analyzed by UHPLC.

In an aspect, where more than one impurity of the individual active pharmaceutical ingredient can be formed over time, a stable composition can be one that has not more than a 1%, 2%, 3%, 4%, or 5% of the individual impurity formation after a predetermined time analyzed by liquid chromatography, e.g., HPLC, UHPLC, LC/MS.

In an aspect, where more than one impurity of the individual active pharmaceutical ingredient can be formed over time, a stable composition can be one that has not more than a 1%, 2%, 3%, 4%, or 5% increase of the individual impurity formation after a predetermined time analyzed by liquid chromatography, e.g., HPLC, UHPLC, LC/MS.

In one aspect, pharmaceutical compositions described herein are stable for at least 14 days at temperature of 40° C.

In one aspect, pharmaceutical compositions described herein are stable for at least 1 month at temperature of 40° C.

In one aspect, a stable composition can be one that has not more than a 2% increase of individual impurity formation after 1 month at temperature of 40° C. analyzed by UHPLC.

In one aspect, a stable composition can be one that has not more than a 2% increase of individual impurity formation after 3 months under controlled room temperature analyzed by UHPLC.

In one aspect, a stable composition can be one that has not more than 2% of individual impurity formation after 3 months under controlled room temperature analyzed by UHPLC.

In one aspect, a stable composition can be one that has not more than a 2% increase of each of oxytocin trisulfide and monodeadmidated oxytocin after 1 month at temperature of 40° C. analyzed by UHPLC.

In one aspect, a stable composition can be one that has not more than 2% each of oxytocin trisulfide and monodeadmidated oxytocin after 1 month at temperature of 40° C. analyzed by UHPLC.

In one aspect, a stable composition can be one that has not more than a 2% increase of oxytocin trisulfide after 1 month at temperature of 40° C. analyzed by UHPLC.

In one aspect, a stable composition can be one that has not more than 2% of oxytocin trisulfide after 1 month at temperature of 40° C. analyzed by UHPLC.

In one aspect, a stable composition can be one that has not more than a 2% increase of monodeadmidated oxytocin after 1 month at temperature of 40° C. analyzed by UHPLC.

In one aspect, a stable composition can be one that has not more than 2% of monodeadmidated oxytocin after 1 month at temperature of 40° C. analyzed by UHPLC.

In one aspect, a stable composition can be one that has not more than a 4% increase of individual impurity formation after 1 month at temperature of 40° C. analyzed by UHPLC.

In one aspect, a stable composition can be one that has not more than a 2% increase of each oxytocin trisulfide and monodeadmidated oxytocin after 3 months under controlled room temperature analyzed by UHPLC.

In one aspect, a stable composition can be one that has not more than a 2% increase of oxytocin trisulfide after 3 months under controlled room temperature analyzed by UHPLC.

In one aspect, a stable composition can be one that has not more than a 2% increase of oxytocin trisulfide after 6 months under controlled room temperature analyzed by UHPLC.

In one aspect, a stable composition can be one that has not more than a 2% increase of monodeadmidated oxytocin after 1 month under controlled room temperature analyzed by UHPLC.

In one aspect, a stable composition can be one that has not more than a 2% increase of monodeadmidated oxytocin after 3 months under controlled room temperature analyzed by UHPLC.

In one aspect, a stable composition can be one that has not more than a 3% increase of monodeadmidated oxytocin after 6 months under controlled room temperature analyzed by UHPLC. In one aspect, a stable composition can be one that has not more than a 3% increase of individual impurity formation after 3 months under controlled room temperature analyzed by UHPLC.

In an aspect, a stable composition can be one that has not more than 1% of dimer impurity when subjected to a temperature of 70° C. for 2 days.

In an aspect, a stable composition can be one that has not more than 2% of dimer impurity when subjected to a temperature of 70° C. for 2 days.

In an aspect, a stable composition can be one that has not more than 2% of dimer impurity when subjected to a temperature of 70° C. for 5 days.

In an aspect, a stable composition can be one that has not more than 3% of dimer impurity when subjected to a temperature of 70° C. for 5 days.

In an aspect, a stable composition can be one that has not more than 4% of dimer impurity when subjected to a temperature of 70° C. for 5 days.

In an aspect, a stable composition can be one that has not more than 6% of dimer impurity when subjected to a temperature of 70° C. for 5 days.

As used herein, “stable” may be defined as no visible particles (free of particles or particles/precipitate free) in the pharmaceutical formulation after a predefined time.

In one aspect, the composition is free of particles when stored for at least 1 month on 40° C.

In one aspect, the composition is free of particles when stored for at least 3 months under controlled room temperature.

By the terms “pharmaceutical composition” or “pharmaceutically acceptable composition” as used herein, is meant any composition suitable and intended for in vivo use, for example administration to a patient or a subject. As used herein, the terms “patient” and “subject” are interchangeable and refer to any human or animal individual who is receiving a composition as described herein.

As used herein, the terms “pharmaceutical composition”, “pharmaceutically acceptable composition”, “pharmaceutical formulation”, “composition” and “formulation” are used interchangeably.

In one aspect, the concentration of oxytocin in the formulation is from 5 mU/ml to 100 mU/ml.

In one aspect, the concentration of oxytocin in the formulation is from 10 mU/ml to 60 mU/ml.

In one aspect, the concentration of oxytocin in the formulation is from 10 mU/ml, 20 mU/ml, 30 mU/ml, 40 mU/ml, 50 mU/ml or 60 mU/ml.

In one aspect, the concentration of oxytocin in the formulation is 60 mU/ml.

In one aspect, oxytocin is in the form of a salt. An oxytocin salt can be selected from succinate, maleate, citrate, tartrate, aspartate, gluconate, and acetate salts. In one aspect, oxytocin is in form of acetate salt.

In one aspect, oxytocin is in form of oxytocin acetate salt hydrate.

In an aspect, the pharmaceutical formulation is aqueous.

By the term “aqueous composition”, “aqueous solution” or “aqueous” is understood any composition in which water is present in or above 50% v/v, such as, e.g., a composition comprising from 50% v/v to 99.5% v/v water, from 50% v/v to 90% v/v, from 60% v/v to 85% v/v, from 70% v/v to 80% v/v water. Accordingly, aqueous compositions include compositions comprising 50% v/v or more, 60% v/v or more, 70% v/v or more, 75% v/v or more, 80% v/v or more, 85% v/v or more, 90% v/v or more, 95% v/v or more, or 99% v/v or more water.

In one aspect, aqueous composition of oxytocin comprises 90% or more water.

In an aspect, pharmaceutical formulation may comprise water or standard diluents for parenteral use, such as water for injection, 0.9% sodium chloride for injection, Sterile water for injection, Dextrose 5% injection or other suitable diluents.

In one aspect, formulation comprises 0.9% sodium chloride for injection.

In one aspect, the compositions of oxytocin comprise at least one cyclodextrin.

In one aspect, the liquid compositions of oxytocin comprise at least one beta cyclodextrin.

In one aspect, exemplary cyclodextrins are 2-hydroxypropyl-beta-cyclodextrin (HPBCD) and sulfobutylether-beta-cyclodextrin (SBEBCD).

In one aspect, the beta cyclodextrin is SBECD.

Sulfobutylether-beta-cyclodextrin (SBECD) is an anionic beta cyclodextrin derivative of a sodium sulfonate salt separated from the hydrophobic cavity by a butyl ether spacer group. The sulfobutylether substituent is introduced at the 2, 3, and 6 positions in one or more of the glucopyranose units in the cyclodextrin structure.

In one aspect, the average degree of substitution of SBECD is from 5.9 to 7.1.

In one aspect, the average degree of substitution of SBECD is 5.9, 6.0, 6.1, 6.2, 6.3, 6.4, 6.5, 6.6, 6.7, 6.8, 6.9, 7.0 or 7.1.

Degree of substitution may be expressed as average degree of substitution, which is understood as number of substituents per cyclodextrin ring. Degree of substitution may also be expressed as a molar substitution, which is number of substituents per one glucose unit of cyclodextrin.

In one aspect, the beta cyclodextrin is HPBCD.

Hydroxypropyl-beta-cyclodextrin is a partially substituted poly(2-hydroxpropyl) ether of beta-cyclodextrin. In one aspect, molar substitution of HPBCD 0.4-1.5.

In one aspect, molar substitution of HPBCD 0.5-1.1.

In another aspect, molar substitution of HPBCD is 0.6-1.0.

In another aspect, molar substitution of HPBCD is 0.6-0.9.

In another aspect, molar substitution of HPBCD is 0.5, 0.6, 0.7, 0.8, 0.9, 1.0 or 1.1.

In one aspect, at least one cyclodextrin is in concentration from 0.01 mM to 5 mM.

In other aspect, at least one cyclodextrin is in concentration from 0.1 mM to 1 mM.

In one aspect, at least one cyclodextrin is in concentration of 0.05 mM, 0.1 mM, 0.2 mM, 0.3 mM, 0.4 mM, 0.5 mM, 0.6 mM, 0.7 mM, 0.8 mM, 0.9 mM or 1.0 mM.

In some aspects, formulation comprises at least one amino acid. The term “amino acid” means any amino acid, including, but is not limited to the 20 amino acids naturally occurring in peptides and is also meant to cover any salt thereof, especially pharmaceutically acceptable salts. For example, the term “amino acid” includes alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and ornithine, and salts thereof.

In one aspect, the amino acid is aspartic acid, serine, glutamic acid or a pharmaceutically acceptable salt thereof.

In one aspect, the formulation comprises at least two amino acids.

In one aspect, the formulation comprises at least three amino acids.

In an aspect, the amino acid is glutamic acid.

In an aspect, the amino acid is serine.

In an aspect, the amino acid is aspartic acid.

In some aspects, the amino acids may be in L configuration, while in some other aspects they may be in D configuration.

In an aspect, the glutamic acid is L-glutamic acid or a pharmaceutically acceptable salt thereof.

In an aspect, the amino acid is L-serine.

In an aspect, the amino acid is L-aspartic acid.

In one aspect, the formulation comprises HPBCD and L-aspartic acid.

In one aspect, the formulation comprises HPBCD and L-serine.

In one aspect, the formulation comprises HPBCD and L-glutamic acid.

In one aspect, the formulation comprises L-glutamic acid and serine.

In one aspect, the formulation comprises L-serine and L-aspartic acid.

In one aspect, the formulation comprises L-glutamic acid and L-aspartic acid.

In one aspect, the concentration of at least one amino acid is from 0.5 mM to 200 mM.

In one aspect, the concentration of at least one amino acid is from 5.5 mM to 50 mM.

In one aspect, the concentration of at least one amino acid is from 5.5 mM to 15 mM.

In one aspect, the concentration of at least one amino acid is 1 mM, 2 mM, 3 mM, 4 mM, 5 mM, 5.5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM or 25 mM.

In one aspect, the concentration of L-glutamic acid is from 0.1 mg/ml to 5 mg/ml.

In one aspect, the concentration of L-glutamic is from 0.8 mg/ml to 2 mg/ml.

In one aspect, oxytocin and at least one amino acid are in molar ratio of 1:0.5 to 1:20.

In one aspect, the oxytocin and at least one amino acid are in molar ratio of 1:0.5, 1:1, 1:1, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20.

In an aspect, the pharmaceutical formulation comprises at least one organic acid or a pharmaceutically acceptable salt thereof, wherein the organic acid is not an amino acid. In an aspect, the at least one organic acid may be selected from aldonic acids, such as lactobionic acid, and gluconic acid; dicarboxylic acids, specifically alpha, omega dicarboxylic acids, such as adipic acid; and/or carboxylic acids such as picolinic acid. In one aspect, the at least one organic acid may be selected from adipic acid, lactobionic acid, gluconic acid, picolinic acid, or a pharmaceutically acceptable salt thereof. Combinations of organic acids may be employed.

In one aspect, the formulation comprises at least organic two acids.

In one aspect, the formulation comprises at least three organic acids.

In one aspect, the formulation comprises aldonic acid or its pharmaceutically acceptable salt.

In one aspect, the formulation comprises gluconic acid or a pharmaceutically acceptable salt thereof.

In one aspect, the formulation comprises sodium gluconate. The term “sodium gluconate” as used herein means D-Gluconic acid, sodium.

In one aspect, the formulation comprises lactobionic acid or a pharmaceutically acceptable salt thereof. The term “lactobionic acid” as used herein means 4-O-β-D-Galactopyranosyl-D-gluconic acid.

In one aspect, the formulation comprises adipic acid.

In one aspect, the formulation comprises HPBCD and sodium gluconate.

In one aspect, the formulation comprises HPBCD and lactobionic acid.

In one aspect, the formulation comprises HPBCD and adipic acid.

In one aspect, the formulation comprises sodium gluconate and lactobionic acid.

In one aspect, the formulation comprises gluconic acid and lactobionic acid or a pharmaceutically acceptable salt thereof.

In one aspect, the formulation comprises gluconic acid and adipic acid or a pharmaceutically acceptable salt thereof.

In one aspect, formulation comprises gluconic acid and L-glutamic acid or a pharmaceutically acceptable salt thereof.

In one aspect, the formulation comprises sodium gluconate and adipic acid.

In one aspect, the formulation comprises sodium gluconate and L-glutamic acid.

In one aspect, the formulation comprises sodium gluconate and L-serine.

In one aspect, the formulation comprises sodium gluconate and L-aspartic acid.

In one aspect, the formulation comprises lactobionic acid and adipic acid.

In one aspect, the formulation comprises lactobionic acid and L-glutamic acid.

In one aspect, the formulation comprises lactobionic acid and L-serine.

In one aspect, the formulation comprises lactobionic acid and L-aspartic acid.

In one aspect, the formulation comprises adipic acid and L-glutamic acid.

In one aspect, the formulation comprises adipic acid and L-serine.

In one aspect, the formulation comprises adipic acid and L-aspartic acid.

In one aspect, the concentration of at least one organic acid is from 0.5 mM to 200 mM.

In one aspect, the concentration of at least one organic acid is from 5.5 mM to 50 mM.

In one aspect, the concentration of at least one organic acid is from 5.5 mM to 15 mM.

In one aspect, the concentration of at least one organic acid is 5.5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM or 25 mM.

In one aspect, the concentration of gluconic acid is from 0.1 mg/ml to 5 mg/ml.

In one aspect, the concentration of gluconic acid is from 1.3 mg/ml to 2 mg/ml.

In one aspect, the concentration of sodium gluconate is from 0.1 mg/ml to 5 mg/ml.

In one aspect, the concentration of sodium gluconate is from 0.8 mg/ml to 2 mg/ml.

In one aspect, the concentration of sodium gluconate is from 1.3 mg/ml to 2 mg/ml.

In one aspect, the concentration of sodium gluconate is from 1.3 mg/ml to 1.5 mg/ml.

In one aspect, the concentration of lactobionic acid is from 0.1 mg/ml to 5 mg/ml.

In one aspect, the concentration of lactobionic is from 0.5 mg/ml to 2 mg/ml.

In one aspect, the formulation comprises magnesium or its pharmaceutically acceptable salt. In one aspect, formulation comprises magnesium chloride.

In an aspect, the pharmaceutical formulation does not comprise a chelating agent.

In an aspect, the pharmaceutical formulation does not comprise EDTA.

In an aspect, the pharmaceutical formulation does not comprise chlorobutanol.

In an aspect, the pharmaceutical formulation does not comprise cyclodextrins.

In an aspect, the pharmaceutical formulation does not comprise sodium acetate.

In an aspect, the pharmaceutical formulation does not comprise magnesium or its pharmaceutically acceptable salt.

The pH of the solutions may be adjusted in any suitable manner. The pH may be adjusted with one or more pH adjusting agents, which may be selected from mineral acids, organic acids, weak and strong bases, and salts and derivatives thereof. Examples of pH adjusting agents include hydrochloric acid, phosphoric acid, sulfuric acid, acetic acid, succinic acid, lactic acid, citric acid, phenolic acid, sodium hydroxide, ammonium hydroxide, sodium bicarbonate, or similar.

In one aspect, the pH is adjusted with sodium hydroxide and hydrochloric acid.

In one aspect, the pH is adjusted with hydrochloric acid.

In one aspect, the formulation is in form of a ready-to-administer composition.

A “ready-to-administer” composition is synonymous with “ready-to-infuse” or “ready-to-inject”. A “ready-to-administer” composition is suitable for administration directly to the patient and does not require any dilution steps.

Ready-to-administer formulations according to the present disclosure may be used by administering oxytocin to a subject in need thereof.

Ready-to-administer formulations according to the present disclosure may be used for initiation or improvement of uterine contractions by administering oxytocin to a subject in need thereof.

In one aspect, the ready-to-administer formulation may be used for induction of labor in patients with a medical indication for the initiation of labor, such as Rh problems, maternal diabetes, preeclampsia at or near term, when delivery is in the best interests of mother and fetus or when membranes are prematurely ruptured, and delivery is indicated.

In one aspect, the ready-to-administer formulation may be used for stimulation or reinforcement of labor, as in selected cases of uterine inertia in a subject.

In one aspect, the ready-to-administer formulation may be used as adjunctive therapy in the management of incomplete or inevitable abortion.

In one aspect, the ready-to-administer formulation may be used to produce uterine contractions during the third stage of labor and to control postpartum bleeding or hemorrhage.

This disclosure also provides a process for manufacturing any disclosed pharmaceutical formulations. In various embodiments, the process may comprise the steps of dissolving predefined excipients and oxytocin in water or predefined standard diluent for parenteral use and filling the predefined container with the formulation. If necessary, pH is adjusted to achieve the desired pH range.

In one aspect, ready-to-administer formulations of oxytocin may be prepared by dissolving excipients in water for injection or predefined standard diluent for parenteral use under predefined conditions until a clear solution is obtained. Oxytocin is added to the solution containing dissolved excipients and the solution is mixed until oxytocin is dissolved. If necessary, pH is adjusted to a predefined pH value, and then WFI or appropriate diluent is added to make up to a predefined volume. The prepared formulation is then mixed to ensure homogeneity, and afterwards filtered through a 0.2 μm filter, and aliquoted in predefined packaging.

In another aspect, pharmaceutical formulation may be manufactured by any process known to the person skilled in the art.

In one aspect, the liquid formulation of oxytocin is packaged into a suitable container. Thus, included herein are products comprising the oxytocin formulations described herein. The container may be a vial, an ampule or a bag. In one aspect, the container is a single unit dose container. In one aspect, the container is a single unit dosage container for IV administration.

In one aspect, the volume of container is from 50 ml to 1000 ml.

In one aspect, the volume of container is from 100 ml to 1000 ml.

In one aspect, the volume of container is 50 ml, 100 ml, 200 ml, 250 ml, 300 ml, 400 ml, 500 ml, 600 ml, 700 ml, 800 ml, 900 ml or 1000 ml.

In one aspect, the volume of container is 500 ml.

In an aspect, the formulation is stored in a container used for intravenous administration, which is preferably plastic based. In an aspect, the formulation is packaged in a flexible plastic bag. In one aspect, formulation is packaged in a container produced by blow-fill-seal technology. In one aspect, the material of the container is PVC free. In one aspect, the formulation is not in contact with the PVC material of the container.

In one aspect, the plastic material may be a polyolefin, such as polyethylene (PE), polypropylene (PP), ethylene vinyl acetate (EVA), ethylene vinyl alcohol (EVOH), a polyolefin/styrene-block copolymer and derivatives thereof with or without other additives, and combinations, in particular blends, composites or laminates, of at least two of said polymeric materials.

In one aspect, the plastic material of the container comprises polyethylene (PE).

In one aspect, the plastic material of the container comprises ethylene vinyl alcohol (EVOH).

In an aspect, the container is a single layer or multilayer container.

In one aspect, the inner layer of the container, which is in contact with the formulation, comprises polyethylene.

In one aspect, the inner layer of the plastic material, which is in contact with the formulation, is polyethylene.

In one aspect, the barrier layer of the plastic material is EVOH.

In an aspect, a ready-to-administer injectable formulation does not require removal of oxygen from the container in which the formulation is stored. That is, oxygen needs not to be removed from the headspace of the container, and the solution needs not to be purged with an inert gas.

In one aspect, the container may, optionally, further comprise an overwrap. In one aspect, the overwrap may comprise gas-barrier and/or light barrier material.

In one aspect, the overwrap is made of aluminum.

In one aspect, the space between a container and an overwrap is filled with inert gas or any other suitable gas. In one aspect, the space between a container and an overwrap is filled with nitrogen (N2), argon or any combination thereof.

In one aspect, the space between a container and an overwrap is vacuumed.

In one aspect, the space between a container and an overwrap comprises an oxygen scavenger.

In one aspect, the container is overwrapped with a water-vapor-barrier overwrap. In one aspect, the container is overwrapped with a water-vapor-barrier overwrap and the space between the container and the overwrap is not filled with nitrogen or any other inert gas. In one aspect, the container is overwrapped with a water-vapor-barrier overwrap and the space between the container and the overwrap is not filled with nitrogen or any other inert gas and does not comprise an oxygen scavenger.

In one aspect, the container is overwrapped with an overwrap and the space between the container and the overwrap is not filled with nitrogen or any other inert gas. In one aspect, the container is overwrapped with an overwrap and the space between the container and the overwrap is not filled with nitrogen or any other inert gas and does not comprise an oxygen scavenger.

In one aspect, the formulation does not need to be overwrapped. Also, the formulation does not require any atmosphere controlling methods for reduction of oxygen content in between the product and the overwrap.

The formulation disclosed herein may be sterilized by known means. Such known means in the art comprise sterile filtration, heat treatment and/or irradiation.

In one aspect, the present disclosure provides a method of treating humans by administering an effective dose of oxytocin by an IV route.

In one aspect, present disclosure provides a method of treating humans by administering a ready-to-administer effective dose of oxytocin formulation by an IV injection.

In an aspect, formulations described herein are intended to be administered via injection or infusion, for example intravenously, or intramuscularly.

In one aspect, the disclosure provides a method of treating humans with an effective dose of oxytocin, the method comprising intravenously administering to the human the ready to administer pharmaceutical composition disclosed herein directly from the unit dosage form, wherein the unit dosage form comprises from 10 mU/ml to 60 mU/ml of oxytocin or a pharmaceutically-acceptable salt thereof.

In one aspect, the disclosure provides a method of treating humans with an effective dose of oxytocin, the method comprising: intravenously administering to the human the ready to administer pharmaceutical composition disclosed herein directly from the unit dosage form, wherein the unit dosage form comprises 60 mU/ml of oxytocin or a pharmaceutically acceptable salt thereof.

All the numbers used herein, except for pH, are modified by the term “about.” This means that each number includes minor variations as defined ±10% of the numerical value or range in question.

In one aspect, the composition is an isosmotic composition. It is to be understood that the term “isosmotic” in accordance with the present disclosure means having similar osmolality to the physiologic osmolality of blood.

Typically, the ready-to-administer pharmaceutical compositions have an osmolality from 250 to 350 mOsm/kg.

In one aspect, the composition has osmolality from 240 to 600 mOsm/kg.

In one aspect, the ready-to-administer formulation included in the ready-to-administer product is both isotonic and has an osmolality similar to the physiological osmolality of blood as described above.

In some aspects, the ready-to-administer pharmaceutical compositions further comprise one or more osmolality adjusting agents. Exemplary osmolality adjusting agents for use in ready-to-administer pharmaceutical compositions include but are not limited to sodium chloride and dextrose.

If not otherwise stated, calculations of molar ratio or concentrations of oxytocin in the present disclosure are done based on oxytocin free base.

If not otherwise stated, calculations of molar ratio or concentrations of excipients in the present disclosure are done based on free base of said excipients.

Additionally, the compositions described herein may further comprise one or more pharmaceutical excipients such as antioxidants, surfactants, complexing agents, preservatives, vehicles, solubilizers, and combinations thereof.

Accordingly, the present disclosure provides a ready-to-administer formulation comprising oxytocin in concentration of 5 mU/ml to 100 mU/ml, gluconic acid or its pharmaceutically acceptable salt, wherein the pH is in the range of 3.2 to 5.5.

The present disclosure provides a ready-to-administer formulation comprising oxytocin in concentration of 5 mU/ml to 100 mU/ml, gluconic acid or its pharmaceutically acceptable salt, wherein the pH is in the range of 3.8 to 4.5.

The present disclosure provides a ready-to-administer formulation comprising oxytocin in concentration of 10 mU/ml to 60 mU/ml, gluconic acid or its pharmaceutically acceptable salt, wherein the pH is in the range of 3.2 to 5.5.

The present disclosure provides a ready-to-administer formulation comprising oxytocin in concentration of 10 mU/ml to 60 mU/ml, gluconic acid or its pharmaceutically acceptable salt, wherein the pH is in the range of 3.8 to 4.5.

The present disclosure provides a ready-to-administer formulation comprising oxytocin in concentration of 5 mU/ml to 100 mU/ml, sodium gluconate, wherein the pH is in the range of 3.2 to 5.5.

The present disclosure provides a ready-to-administer formulation comprising oxytocin in concentration of 5 mU/ml to 100 mU/ml, sodium gluconate, wherein the pH is in the range of 3.8 to 4.5.

The present disclosure provides a ready-to-administer formulation comprising oxytocin in concentration of 10 mU/ml to 60 mU/ml, sodium gluconate, wherein the pH is in the range of 3.2 to 5.5.

The present disclosure provides a ready-to-administer formulation comprising oxytocin in concentration of 10 mU/ml to 60 mU/ml, sodium gluconate, wherein the pH is in the range of 3.6 to 4.5.

The present disclosure provides a ready-to-administer formulation comprising oxytocin in concentration of 10 mU/ml to 60 mU/ml, sodium gluconate, wherein the pH is in the range of 3.8 to 4.5.

The present disclosure provides a ready-to-administer formulation comprising 5 mU/ml to 100 mU/ml oxytocin, gluconic acid or its pharmaceutically acceptable salt and where gluconic acid or its pharmaceutically acceptable salt is in concentration from 0.1 mg/ml to 5 mg/ml, and where the pH is in the range of 3.2 to 5.5.

The present disclosure provides a ready-to-administer formulation comprising 5 mU/ml to 100 mU/ml oxytocin, gluconic acid or its pharmaceutically acceptable salt and where gluconic acid or its pharmaceutically acceptable salt is in concentration from 0.1 mg/ml to 5 mg/ml, and where the pH is in the range of 3.8 to 4.5.

The present disclosure provides a ready-to-administer formulation comprising oxytocin in concentration of 10 mU/ml to 60 mU/ml, gluconic acid or its pharmaceutically acceptable salt and where gluconic acid or its pharmaceutically acceptable salt is in concentration from 0.1 mg/ml to 5 mg/ml, and wherein the pH is in the range of 3.2 to 5.5.

The present disclosure provides a ready-to-administer formulation comprising oxytocin in concentration of 10 mU/ml to 60 mU/ml, gluconic acid or its pharmaceutically acceptable salt and where gluconic acid or its pharmaceutically acceptable salt is in concentration from 0.1 mg/ml to 5 mg/ml, wherein the pH is in the range of 3.8 to 4.5.

The present disclosure provides a ready-to-administer formulation comprising oxytocin in concentration of 5 mU/ml to 100 mU/ml, sodium gluconate and where sodium gluconate is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, sodium gluconate and where sodium gluconate is in concentration from 0.1 mg/ml to 5 mg/ml, wherein the pH is in the range of 3.8 to 4.5.

The present disclosure provides a ready-to-administer formulation comprising oxytocin in concentration of 10 mU/ml to 60 mU/ml, sodium gluconate and where sodium gluconate is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, sodium gluconate and where sodium gluconate is in concentration from 0.1 mg/ml to 5 mg/ml, wherein the pH is in the range of 3.8 to 4.5.

The present disclosure provides a ready-to-administer formulation comprising 5 mU/ml to 100 mU/ml oxytocin, gluconic acid or its pharmaceutically acceptable salt and where gluconic acid or its pharmaceutically acceptable salt is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, gluconic acid or its pharmaceutically acceptable salt and where gluconic acid or its pharmaceutically acceptable salt is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, gluconic acid or its pharmaceutically acceptable salt and where gluconic acid or its pharmaceutically acceptable salt is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, gluconic acid or its pharmaceutically acceptable salt and where gluconic acid or its pharmaceutically acceptable salt is in concentration from 0.8 mg/ml to 2 mg/ml, wherein the pH is in the range of 3.8 to 4.5.

The present disclosure provides a ready-to-administer formulation comprising oxytocin in concentration of 5 mU/ml to 100 mU/ml, sodium gluconate and where sodium gluconate is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, sodium gluconate and where sodium gluconate is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, sodium gluconate and where sodium gluconate is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, sodium gluconate and where sodium gluconate is in concentration from 0.8 mg/ml to 2 mg/ml, wherein the pH is in the range of 3.6 to 4.5.

The present disclosure provides a ready-to-administer formulation comprising oxytocin in concentration of 10 mU/ml to 60 mU/ml, sodium gluconate and where sodium gluconate is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, sodium gluconate and where sodium gluconate is in concentration from 0.8 mg/ml to 2 mg/ml, wherein the pH is in the range of 3.6 to 4.5, where the formulation is stable at least 3 months under controlled room temperature having no more than 8% drop of oxytocin assay as determined by UHPLC.

The present disclosure provides a ready-to-administer formulation comprising oxytocin in concentration of 10 mU/ml to 60 mU/ml, sodium gluconate and where sodium gluconate is in concentration from 0.8 mg/ml to 2 mg/ml, wherein the pH is in the range of 3.8 to 4.5, where the formulation is stable at least 3 months under controlled room temperature having no more than 8% drop of oxytocin assay as determined by UHPLC.

The present disclosure provides a ready-to-administer formulation, essentially consisting of oxytocin in concentration of 10 mU/ml to 60 mU/ml, 0.8 mg/ml to 2 mg/ml sodium gluconate, sodium chloride 9 mg/ml, optionally pH adjusting agent and water and where pH is in the range of 3.8 to 4.5.

The present disclosure provides a ready-to-administer formulation consisting of oxytocin in concentration of 10 mU/ml to 60 mU/ml, 0.8 mg/ml to 2 mg/ml of sodium gluconate, sodium chloride 9 mg/ml, optionally pH adjusting agent and water and where pH is in the range of 3.6 to 4.5.

The present disclosure provides a ready-to-administer formulation consisting of 10 mU/ml to 60 mU/ml of oxytocin, 0.8 mg/ml to 2 mg/ml of sodium gluconate, sodium chloride 9 mg/ml, optionally pH adjusting agent and water and where pH is in the range of 3.8 to 4.5.

Accordingly, the present disclosure provides a ready-to-administer formulation comprising 5 mU/ml to 100 mU/ml of oxytocin, L-glutamic acid or its pharmaceutically acceptable salt, wherein the pH is in the range of 3.2 to 5.5.

The present disclosure provides a ready-to-administer formulation comprising 5 mU/ml to 100 mU/ml oxytocin, L-glutamic or its pharmaceutically acceptable salt, wherein the pH is in the range of 3.8 to 4.5.

The present disclosure provides a ready-to-administer formulation comprising oxytocin in concentration of 10 mU/ml to 60 mU/ml, L-glutamic acid or its pharmaceutically acceptable salt, wherein the pH is in the range of 3.2 to 5.5.

The present disclosure provides a ready-to-administer formulation comprising oxytocin in concentration of 10 mU/ml to 60 mU/ml, L-glutamic acid or its pharmaceutically acceptable salt, wherein the pH is in the range of 3.8 to 4.5.

The present disclosure provides a ready-to-administer formulation comprising oxytocin in concentration of 5 mU/ml to 100 mU/ml, L-glutamic acid, wherein the pH is in the range of 3.2 to 5.5.

The present disclosure provides a ready-to-administer formulation comprising 5 mU/ml to 100 mU/ml oxytocin, L-glutamic acid, wherein the pH is in the range of 3.8 to 4.5.

The present disclosure provides a ready-to-administer formulation comprising oxytocin in concentration of 10 mU/ml to 60 mU/ml, L-glutamic acid, wherein the pH is in the range of 3.2 to 5.5.

The present disclosure provides a ready-to-administer formulation comprising oxytocin in concentration of 10 mU/ml to 60 mU/ml, L-glutamic acid, wherein the pH is in the range of 3.8 to 4.5.

The present disclosure provides a ready-to-administer formulation comprising 5 mU/ml to 100 mU/ml oxytocin, L-glutamic acid or its pharmaceutically acceptable salt and where L-glutamic acid or its pharmaceutically acceptable salt is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, L-glutamic acid or its pharmaceutically acceptable salt and where L-L-glutamic acid or its pharmaceutically acceptable salt is in concentration from 0.1 mg/ml to 5 mg/ml, wherein the pH is in the range of 3.8 to 4.5.

The present disclosure provides a ready-to-administer formulation comprising oxytocin in concentration of 10 mU/ml to 60 mU/ml, L-glutamic acid or its pharmaceutically acceptable salt and where L-glutamic acid or its pharmaceutically acceptable salt is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, L-glutamic acid or its pharmaceutically acceptable salt and where L-glutamic acid or its pharmaceutically acceptable salt is in concentration from 0.1 mg/ml to 5 mg/ml, wherein the pH is in the range of 3.8 to 4.5.

The present disclosure provides a ready-to-administer formulation comprising oxytocin in concentration of 5 mU/ml to 100 mU/ml, L-glutamic acid and where L-glutamic acid is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, L-glutamic acid and where L-glutamic acid is in concentration from 0.1 mg/ml to 5 mg/ml, wherein the pH is in the range of 3.8 to 4.5.

The present disclosure provides a ready-to-administer formulation comprising oxytocin in concentration of 10 mU/ml to 60 mU/ml, L-glutamic acid and where L-glutamic acid is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, L-glutamic acid and where L-glutamic acid is in concentration from 0.1 mg/ml to 5 mg/ml, wherein the pH is in the range of 3.8 to 4.5.

The present disclosure provides a ready-to-administer formulation comprising 5 mU/ml to 100 mU/ml oxytocin, L-glutamic acid or its pharmaceutically acceptable salt and where L-glutamic acid or its pharmaceutically acceptable salt is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, L-glutamic acid or its pharmaceutically acceptable salt and where L-glutamic acid or its pharmaceutically acceptable salt is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, L-glutamic acid or its pharmaceutically acceptable salt and where L-glutamic acid or its pharmaceutically acceptable salt is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, L-glutamic acid or its pharmaceutically acceptable salt and where L-glutamic acid or its pharmaceutically acceptable salt is in concentration from 0.8 mg/ml to 2 mg/ml, wherein the pH is in the range of 3.8 to 4.5.

The present disclosure provides a ready-to-administer formulation comprising oxytocin in concentration of 5 mU/ml to 100 mU/ml, L-glutamic acid and where L-glutamic acid is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, L-glutamic acid and where L-glutamic acid is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, L-glutamic acid and where L-glutamic acid is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, L-glutamic acid and where L-glutamic acid is in concentration from 0.8 mg/ml to 2 mg/ml, wherein the pH is in the range of 3.6 to 5.5.

The present disclosure provides a ready-to-administer formulation comprising oxytocin in concentration of 10 mU/ml to 60 mU/ml, L-glutamic acid and where L-glutamic acid is in concentration from 0.8 mg/ml to 2 mg/ml, wherein the pH is in the range of 3.8 to 5.5.

The present disclosure provides a ready-to-administer formulation comprising oxytocin in concentration of 10 mU/ml to 60 mU/ml, L-glutamic acid and where L-glutamic acid is in concentration from 0.8 mg/ml to 2 mg/ml, where the pH is in the range of 3.6 to 5.5, where the formulation is stable at least 3 months under controlled room temperature having no more than 8% drop of oxytocin assay as determined by UHPLC.

The present disclosure provides a ready-to-administer formulation comprising oxytocin in concentration of 10 mU/ml to 60 mU/ml, L-glutamic acid and where L-glutamic acid is in concentration from 0.8 mg/ml to 2 mg/ml, where the pH is in the range of 3.8 to 5.5, where the formulation is stable at least 3 months under controlled room temperature having no more than 8% drop of oxytocin assay as determined by UHPLC.

The present disclosure provides a ready-to-administer formulation, essentially consisting of oxytocin in concentration of 10 mU/ml to 60 mU/ml, 0.8 mg/ml to 2 mg/ml L-glutamic acid, sodium chloride 9 mg/ml, optionally pH adjusting agent and water and where pH is in the range of 3.8 to 4.5.

The present disclosure provides a ready-to-administer formulation, consisting of oxytocin in concentration of 10 mU/ml to 60 mU/ml, 0.8 mg/ml to 2 mg/ml L-glutamic acid, sodium chloride 9 mg/ml, optionally pH adjusting agent and water and where pH is in the range of 3.6 to 4.5.

The present disclosure provides a ready-to-administer formulation, consisting of oxytocin in concentration of 10 mU/ml to 60 mU/ml, 0.8 mg/ml to 2 mg/ml L-glutamic acid, sodium chloride 9 mg/ml, optionally pH adjusting agent and water and where pH is in the range of 3.8 to 4.5.

Accordingly, the present disclosure provides a ready-to-administer formulation comprising 5 mU/ml to 100 mU/ml oxytocin, L-glutamic acid and sodium gluconate, wherein the pH is in the range of 3.2 to 5.5.

The present disclosure provides a ready-to-administer formulation comprising 5 mU/ml to 100 mU/ml oxytocin, L-glutamic acid and sodium gluconate, wherein the pH is in the range of 3.8 to 5.5.

The present disclosure provides a ready-to-administer formulation comprising oxytocin in concentration of 10 mU/ml to 60 mU/ml, L-glutamic acid and sodium gluconate, wherein the pH is in the range of 3.2 to 5.5.

The present disclosure provides a ready-to-administer formulation comprising oxytocin in concentration of 10 mU/ml to 60 mU/ml, L-glutamic acid and sodium gluconate, wherein the pH is in the range of 3.8 to 5.5.

The present disclosure provides a ready-to-administer formulation comprising 5 mU/ml to 100 mU/ml oxytocin, L-glutamic acid and sodium gluconate and where each of L-glutamic acid and sodium gluconate is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, L-glutamic acid and sodium gluconate and where each of L-glutamic acid and sodium gluconate is in concentration from 0.1 mg/ml to 5 mg/ml, wherein the pH is in the range of 3.8 to 5.5.

The present disclosure provides a ready-to-administer formulation comprising oxytocin in concentration of 10 mU/ml to 60 mU/ml, L-glutamic acid and sodium gluconate and where each of L-glutamic acid and sodium gluconate is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, L-glutamic acid and sodium gluconate and where each of L-glutamic acid and sodium gluconate is in concentration from 0.1 mg/ml to 5 mg/ml, wherein the pH is in the range of 3.8 to 5.5.

The present disclosure provides a ready-to-administer formulation comprising 5 mU/ml to 100 mU/ml oxytocin, L-glutamic acid and sodium gluconate and where each of L-glutamic acid and sodium gluconate is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, L-glutamic acid and sodium gluconate and where each of L-glutamic acid and sodium gluconate is in concentration from 0.8 mg/ml to 2 mg/ml, wherein the pH is in the range of 3.8 to 5.5.

The present disclosure provides a ready-to-administer formulation comprising oxytocin in concentration of 10 mU/ml to 60 mU/ml, L-glutamic acid and sodium gluconate and where each of L-glutamic acid and sodium gluconate is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, L-glutamic acid and sodium gluconate and where each of L-glutamic acid and sodium gluconate is in concentration from 0.8 mg/ml to 2 mg/ml, wherein the pH is in the range of 3.6 to 5.5, where the formulation is stable at least 3 months under controlled room temperature having no more than an 8% drop of oxytocin assay as determined by UHPLC.

The present disclosure provides a ready-to-administer formulation comprising oxytocin in concentration of 10 mU/ml to 60 mU/ml, L-glutamic acid and sodium gluconate and where each of L-glutamic acid and sodium gluconate is in concentration from 0.8 mg/ml to 2 mg/ml, wherein the pH is in the range of 3.8 to 5.5, where the formulation is stable at least 3 months under controlled room temperature having no more than an 8% drop of oxytocin assay as determined by UHPLC.

The present disclosure provides a ready-to-administer formulation, essentially consisting of oxytocin in concentration of 10 mU/ml to 60 mU/ml, 0.8 mg/ml to 2 mg/ml L-glutamic acid, 0.8 mg/ml to 2 mg/ml sodium gluconate, sodium chloride 9 mg/ml, optionally pH adjusting agent and water and where pH is in the range of 3.8 to 4.5.

The present disclosure provides a ready-to-administer formulation, consisting of oxytocin in concentration of 10 mU/ml to 60 mU/ml, 0.8 mg/ml to 2 mg/ml L-glutamic acid, 0.8 mg/ml to 2 mg/ml L-glutamic acid, sodium chloride 9 mg/ml, optionally pH adjusting agent and water and where pH is in the range of 3.6 to 4.5.

The present disclosure provides a ready-to-administer formulation, consisting of oxytocin in concentration of 10 mU/ml to 60 mU/ml, 0.8 mg/ml to 2 mg/ml L-glutamic acid, 0.8 mg/ml to 2 mg/ml L-glutamic acid, sodium chloride 9 mg/ml, optionally pH adjusting agent and water and where pH is in the range of 3.8 to 4.5.

Accordingly, the present disclosure provides a ready-to-administer formulation comprising 5 mU/ml to 100 mU/ml oxytocin, lactobionic acid or its pharmaceutically acceptable salt, wherein the pH is in the range of 3.2 to 5.5.

The present disclosure provides a ready-to-administer formulation comprising 5 mU/ml to 100 mU/ml oxytocin, lactobionic acid or its pharmaceutically acceptable salt, wherein the pH is in the range of 3.8 to 5.5.

The present disclosure provides a ready-to-administer formulation comprising oxytocin in concentration of 10 mU/ml to 60 mU/ml, lactobionic acid or its pharmaceutically acceptable salt, wherein the pH is in the range of 3.2 to 5.5.

The present disclosure provides a ready-to-administer formulation comprising oxytocin in concentration of 10 mU/ml to 60 mU/ml, lactobionic acid or its pharmaceutically acceptable salt, wherein the pH is in the range of 3.8 to 5.5.

The present disclosure provides a ready-to-administer formulation comprising oxytocin in concentration of 5 mU/ml to 100 mU/ml, lactobionic acid, wherein the pH is in the range of 3.2 to 5.5.

The present disclosure provides a ready-to-administer formulation comprising 5 mU/ml to 100 mU/ml oxytocin, lactobionic acid, wherein the pH is in the range of 3.8 to 5.5.

The present disclosure provides a ready-to-administer formulation comprising oxytocin in concentration of 10 mU/ml to 60 mU/ml, lactobionic acid, wherein the pH is in the range of 3.2 to 5.5.

The present disclosure provides a ready-to-administer formulation comprising oxytocin in concentration of 10 mU/ml to 60 mU/ml, lactobionic acid, wherein the pH is in the range of 3.8 to 5.5.

The present disclosure provides a ready-to-administer formulation comprising 5 mU/ml to 100 mU/ml oxytocin, lactobionic acid or its pharmaceutically acceptable salt and where lactobionic acid or its pharmaceutically acceptable salt is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, lactobionic acid or its pharmaceutically acceptable salt and where lactobionic acid or its pharmaceutically acceptable salt is in concentration from 0.1 mg/ml to 5 mg/ml, wherein the pH is in the range of 3.8 to 5.5.

The present disclosure provides a ready-to-administer formulation comprising oxytocin in concentration of 10 mU/ml to 60 mU/ml, lactobionic acid or its pharmaceutically acceptable salt and where lactobionic acid or its pharmaceutically acceptable salt is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, lactobionic acid or its pharmaceutically acceptable salt and where lactobionic acid or its pharmaceutically acceptable salt is in concentration from 0.1 mg/ml to 5 mg/ml, wherein the pH is in the range of 3.8 to 5.5.

The present disclosure provides a ready-to-administer formulation comprising oxytocin in concentration of 5 mU/ml to 100 mU/ml, lactobionic acid and where lactobionic acid is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, lactobionic acid and where lactobionic acid is in concentration from 0.1 mg/ml to 5 mg/ml, wherein the pH is in the range of 3.8 to 5.5.

The present disclosure provides a ready-to-administer formulation comprising oxytocin in concentration of 10 mU/ml to 60 mU/ml, lactobionic acid and where lactobionic acid is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, lactobionic acid and where lactobionic acid is in concentration from 0.1 mg/ml to 5 mg/ml, wherein the pH is in the range of 3.8 to 5.5.

The present disclosure provides a ready-to-administer formulation comprising 5 mU/ml to 100 mU/ml oxytocin, lactobionic acid or its pharmaceutically acceptable salt and where lactobionic acid or its pharmaceutically acceptable salt is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, lactobionic acid or its pharmaceutically acceptable salt and where lactobionic acid or its pharmaceutically acceptable salt is in concentration from 0.8 mg/ml to 2 mg/ml, wherein the pH is in the range of 3.8 to 5.5.

The present disclosure provides a ready-to-administer formulation comprising oxytocin in concentration of 10 mU/ml to 60 mU/ml, lactobionic acid or its pharmaceutically acceptable salt and where lactobionic acid or its pharmaceutically acceptable salt is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, lactobionic acid or its pharmaceutically acceptable salt and where lactobionic acid or its pharmaceutically acceptable salt is in concentration from 0.8 mg/ml to 2 mg/ml, wherein the pH is in the range of 3.8 to 5.5.

The present disclosure provides a ready-to-administer formulation comprising oxytocin in concentration of 5 mU/ml to 100 mU/ml, lactobionic acid and where lactobionic acid is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, lactobionic acid and where lactobionic acid is in concentration from 0.8 mg/ml to 2 mg/ml, wherein the pH is in the range of 3.6 to 5.5.

The present disclosure provides a ready-to-administer formulation comprising 5 mU/ml to 100 mU/ml oxytocin, lactobionic acid and where lactobionic acid is in concentration from 0.8 mg/ml to 2 mg/ml, wherein the pH is in the range of 3.8 to 5.5.

The present disclosure provides a ready-to-administer formulation comprising 5 mU/ml to 100 mU/ml oxytocin, lactobionic acid and where lactobionic acid is in concentration from 0.8 mg/ml to 2 mg/ml, wherein the pH is in the range of 3.8 to 5.5.

The present disclosure provides a ready-to-administer formulation comprising oxytocin in concentration of 10 mU/ml to 60 mU/ml, lactobionic acid and where lactobionic acid is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, lactobionic acid and where lactobionic acid is in concentration from 0.8 mg/ml to 2 mg/ml, wherein the pH is in the range of 3.6 to 5.5.

The present disclosure provides a ready-to-administer formulation comprising oxytocin in concentration of 10 mU/ml to 60 mU/ml, lactobionic acid and where lactobionic acid is in concentration from 0.8 mg/ml to 2 mg/ml, wherein the pH is in the range of 3.8 to 5.5.

The present disclosure provides a ready-to-administer formulation comprising oxytocin in concentration of 10 mU/ml to 60 mU/ml, lactobionic acid and where lactobionic acid is in concentration from 0.8 mg/ml to 2 mg/ml, wherein the pH is in the range of 3.6 to 5.5, where the formulation is stable at least 3 months under controlled room temperature having no more than an 8% drop of oxytocin assay as determined by UHPLC.

The present disclosure provides a ready-to-administer formulation comprising oxytocin in concentration of 10 mU/ml to 60 mU/ml, lactobionic acid and where lactobionic acid is in concentration from 0.8 mg/ml to 2 mg/ml, wherein the pH is in the range of 3.8 to 5.5, where the formulation is stable at least 3 months under controlled room temperature having no more than an 8% drop of oxytocin assay as determined by UHPLC.

The present disclosure provides a ready-to-administer formulation, essentially consisting of oxytocin in concentration of 10 mU/ml to 60 mU/ml, 0.8 mg/ml to 2 mg/ml lactobionic acid, sodium chloride 9 mg/ml, optionally pH adjusting agent and water and where pH is in the range of 3.8 to 4.5.

The present disclosure provides a ready-to-administer formulation, consisting of oxytocin in concentration of 10 mU/ml to 60 mU/ml, 0.8 mg/ml to 2 mg/ml lactobionic acid, sodium chloride 9 mg/ml, optionally pH adjusting agent and water and where pH is in the range of 3.6 to 4.5.

The present disclosure provides a ready-to-administer formulation, consisting of oxytocin in concentration of 10 mU/ml to 60 mU/ml, 0.8 mg/ml to 2 mg/ml lactobionic acid, sodium chloride 9 mg/ml, optionally pH adjusting agent and water and where pH is in the range of 3.8 to 4.5.

Accordingly, the present disclosure provides a ready-to-administer formulation comprising 5 mU/ml to 100 mU/ml oxytocin, L-glutamic acid and lactobionic acid, wherein the pH is in the range of 3.2 to 5.5.

The present disclosure provides a ready-to-administer formulation comprising 5 mU/ml to 100 mU/ml oxytocin, L-glutamic acid and lactobionic acid, wherein the pH is in the range of 3.8 to 5.5.

The present disclosure provides a ready-to-administer formulation comprising oxytocin in concentration of 10 mU/ml to 60 mU/ml, L-glutamic acid and sodium gluconate, wherein the pH is in the range of 3.2 to 5.5.

The present disclosure provides a ready-to-administer formulation comprising oxytocin in concentration of 10 mU/ml to 60 mU/ml, L-glutamic acid and lactobionic acid, wherein the pH is in the range of 3.8 to 5.5.

The present disclosure provides a ready-to-administer formulation comprising 5 mU/ml to 100 mU/ml oxytocin, L-glutamic acid and lactobionic acid and where each of L-glutamic acid and lactobionic acid is in concentration from 0.1 mg/ml to 5 mg/ml, wherein the pH is in the range of 3.2 to 5.5.

The present disclosure provides a ready-to-administer formulation comprising 5 mU/ml to 100 mU/ml oxytocin, L-glutamic acid and lactobionic acid and where each of L-glutamic acid and lactobionic acid is in concentration is from 0.1 mg/ml to 5 mg/ml, wherein the pH is in the range of 3.8 to 5.5.

The present disclosure provides a ready-to-administer formulation comprising oxytocin in concentration of 10 mU/ml to 60 mU/ml, L-glutamic acid and lactobionic acid and where each of L-L-glutamic acid and lactobionic acid is in concentration from 0.1 mg/ml to 5 mg/ml, wherein the pH is in the range of 3.2 to 5.5.

The present disclosure provides a ready-to-administer formulation comprising oxytocin in concentration of 10 mU/ml to 60 mU/ml, L-glutamic acid and lactobionic acid and where each of L-glutamic acid and lactobionic acid is in concentration from 0.1 mg/ml to 5 mg/ml, wherein the pH is in the range of 3.8 to 5.5.

The present disclosure provides a ready-to-administer formulation comprising 5 mU/ml to 100 mU/ml oxytocin, L-glutamic acid and lactobionic acid and where each of L-glutamic acid and lactobionic acid is in concentration from 0.8 mg/ml to 2 mg/ml, wherein the pH is in the range of 3.2 to 5.5.

The present disclosure provides a ready-to-administer formulation comprising 5 mU/ml to 100 mU/ml oxytocin, L-glutamic acid and lactobionic acid and where each of L-glutamic acid and lactobionic acid is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, L-glutamic acid and lactobionic acid and where each of L-glutamic acid and lactobionic acid is in concentration from 0.8 mg/ml to 2 mg/ml, wherein the pH is in the range of 3.2 to 5.5.

The present disclosure provides a ready-to-administer formulation comprising oxytocin in concentration of 10 mU/ml to 60 mU/ml, L-glutamic acid and lactobionic acid and where each of L-glutamic acid and lactobionic acid is in concentration from 0.8 mg/ml to 2 mg/ml, wherein the pH is in the range of 3.6 to 4.5.

The present disclosure provides a ready-to-administer formulation comprising oxytocin in concentration of 10 mU/ml to 60 mU/ml, L-glutamic acid and lactobionic acid and where each of L-glutamic acid and lactobionic acid is in concentration from 0.8 mg/ml to 2 mg/ml, wherein the pH is in the range of 3.8 to 4.5.

The present disclosure provides a ready-to-administer formulation comprising oxytocin in concentration of 10 mU/ml to 60 mU/ml, L-glutamic acid and lactobionic acid and where each of L-glutamic acid and lactobionic acid is in concentration from 0.8 mg/ml to 2 mg/ml, wherein the pH is in the range of 3.6 to 4.5, where the formulation is stable at least 3 months under controlled room temperature having no more than 8% drop of oxytocin assay as determined by UHPLC.

The present disclosure provides a ready-to-administer formulation comprising oxytocin in concentration of 10 mU/ml to 60 mU/ml, L-glutamic acid and lactobionic acid and where each of L-glutamic acid and lactobionic acid is in concentration from 0.8 mg/ml to 2 mg/ml, wherein the pH is in the range of 3.8 to 4.5, where the formulation is stable at least 3 months under controlled room temperature having no more than 8% drop of oxytocin assay as determined by UHPLC.

The present disclosure provides a ready-to-administer formulation, essentially consisting of oxytocin in concentration of 10 mU/ml to 60 mU/ml, 0.8 mg/ml to 2 mg/ml sodium gluconate, 0.8 mg/ml to 2 mg/ml lactobionic acid, sodium chloride 9 mg/ml, optionally pH adjusting agent and water and where pH is in the range of 3.8 to 4.5.

The present disclosure provides a ready-to-administer formulation, consisting of oxytocin in concentration of 10 mU/ml to 60 mU/ml, 0.8 mg/ml to 2 mg/ml L-glutamic acid, 0.8 mg/ml to 2 mg/ml lactobionic acid, sodium chloride 9 mg/ml, optionally pH adjusting agent and water and where pH is in the range of 3.6 to 4.5.

The present disclosure provides a ready-to-administer formulation, consisting of oxytocin in concentration of 10 mU/ml to 60 mU/ml, 0.8 mg/ml to 2 mg/ml L-glutamic acid, 0.8 mg/ml to 2 mg/ml lactobionic acid, sodium chloride 9 mg/ml, optionally pH adjusting agent and water and where pH is in the range of 3.8 to 4.5.

The present disclosure provides a ready-to-administer formulation in a plastic container, formulation comprising oxytocin in concentration of 10 mU/ml to 60 mU/ml, sodium gluconate, wherein the pH is in the range of 3.8 to 4.5.

The present disclosure provides a ready-to-administer formulation in a plastic container, formulation comprising oxytocin in concentration of 10 mU/ml to 60 mU/ml, sodium gluconate in concentration from 0.8 mg/ml to 2 mg/ml, wherein the pH is in the range of 3.8 to 4.5.

The present disclosure provides a ready-to-administer formulation in a plastic bag, formulation comprising oxytocin in concentration of 10 mU/ml to 60 mU/ml, sodium gluconate in concentration from 0.8 mg/ml to 2 mg/ml, wherein the pH of the formulation is in the range of 3.8 to 4.5, and where the plastic bag is overwrapped.

The present disclosure provides a ready-to-administer formulation in a plastic bag, formulation comprising oxytocin in concentration of 10 mU/ml to 60 mU/ml, sodium gluconate in concentration from 0.8 mg/ml to 2 mg/ml, wherein the pH of the formulation is in the range of 3.8 to 4.5, and where the plastic bag is overwrapped and were the space between the plastic bag and the overwrap is not filled with nitrogen.

The present disclosure provides a ready-to-administer formulation in a plastic bag, formulation comprising oxytocin in concentration of 10 mU/ml to 60 mU/ml, sodium gluconate is in concentration from 0.8 mg/ml to 2 mg/ml, wherein the pH of the formulation is in the range of 3.8 to 4.5, and where the plastic bag comprises EVOH barrier, plastic bag is overwrapped and were the space between the plastic bag and the overwrap is not filled with nitrogen.

The present disclosure provides a ready-to-administer formulation in a plastic bag, formulation comprising oxytocin in concentration of 10 mU/ml to 60 mU/ml, sodium gluconate, wherein the pH is in the range of 3.8 to 4.5, and where the plastic bag comprises EVOH barrier.

The present disclosure provides a ready-to-administer formulation in a plastic bag, formulation comprising 10 mU/ml to 60 mU/ml oxytocin, sodium gluconate is in concentration from 0.1 mg/ml to 5 mg/ml, wherein the pH is in the range of 3.8 to 4.5, and where the plastic bag comprises EVOH barrier.

The present disclosure provides a ready-to-administer formulation in a plastic bag, formulation comprising oxytocin in concentration of 10 mU/ml to 60 mU/ml, sodium gluconate is in concentration from 0.8 mg/ml to 2 mg/ml, wherein the pH is in the range of 3.8 to 4.5, and where the plastic container bag EVOH barrier.

The present disclosure provides a ready-to-administer formulation in a plastic container, formulation comprising oxytocin in concentration of 10 mU/ml to 60 mU/ml, sodium gluconate and L-glutamic acid, wherein the pH is in the range of 3.8 to 4.5.

The present disclosure provides a ready-to-administer formulation in a plastic container, formulation comprising oxytocin in concentration of 10 mU/ml to 60 mU/ml, sodium gluconate and L-glutamic acid, where sodium gluconate and L-glutamic acid are each in concentration from 0.8 mg/ml to 2 mg/ml, wherein the pH is in the range of 3.8 to 4.5.

The present disclosure provides a ready-to-administer formulation in a plastic bag, formulation comprising oxytocin in concentration of 10 mU/ml to 60 mU/ml, sodium gluconate and L-glutamic acid, where sodium gluconate and L-glutamic acid are each in concentration from 0.8 mg/ml to 2 mg/ml, wherein the pH is in the range of 3.8 to 4.5, and where the plastic bag is overwrapped.

The present disclosure provides a ready-to-administer formulation in a plastic bag, formulation comprising oxytocin in concentration of 10 mU/ml to 60 mU/ml, sodium gluconate and L-glutamic acid, where sodium gluconate and L-glutamic acid are each in concentration from 0.8 mg/ml to 2 mg/ml, wherein the pH is in the range of 3.8 to 4.5, and where the plastic bag is overwrapped and were the space between the plastic bag and the overwrap is not filled with nitrogen.

The present disclosure provides a ready-to-administer formulation in a plastic bag, formulation comprising oxytocin in concentration of 10 mU/ml to 60 mU/ml, sodium gluconate and L-glutamic acid, where sodium gluconate and L-glutamic acid are each in concentration from 0.8 mg/ml to 2 mg/ml, wherein the pH is in the range of 3.8 to 5.5, and where the plastic bag comprises EVOH barrier, plastic bag is overwrapped and were the space between the plastic bag and the overwrap is not filled with nitrogen.

The present disclosure provides a ready-to-administer formulation in a plastic bag, formulation comprising oxytocin in concentration of 10 mU/ml to 60 mU/ml, sodium gluconate and L-glutamic acid, wherein the pH is in the range of 3.8 to 4.5, and where the plastic bag comprises EVOH barrier.

The present disclosure provides a ready-to-administer formulation in a plastic bag, formulation comprising 10 mU/ml to 60 mU/ml oxytocin, sodium gluconate and L-glutamic acid, where sodium gluconate and L-glutamic acid are each in concentration from 0.1 mg/ml to 5 mg/ml, wherein the pH is in the range of 3.8 to 4.5 and where the plastic bag comprises EVOH barrier.

The present disclosure provides a ready-to-administer formulation in a plastic bag, formulation comprising oxytocin in concentration of 10 mU/ml to 60 mU/ml, sodium gluconate and L-glutamic acid, where sodium gluconate and L-glutamic acid are each in concentration from 0.8 mg/ml to 2 mg/ml, wherein the pH is in the range of 3.8 to 5.5, and where the plastic container bag EVOH barrier.

The present disclosure provides a ready-to-administer formulation in a plastic container, formulation comprising oxytocin in concentration of 10 mU/ml to 60 mU/ml, L-glutamic acid and lactobionic acid, wherein the pH is in the range of 3.8 to 5.5.

The present disclosure provides a ready-to-administer formulation in a plastic container, formulation comprising oxytocin in concentration of 10 mU/ml to 60 mU/ml, L-glutamic acid and lactobionic, where L-glutamic acid and lactobionic acid are each in concentration from 0.8 mg/ml to 2 mg/ml, wherein the pH is in the range of 3.8 to 5.5.

The present disclosure provides a ready-to-administer formulation in a plastic bag, formulation comprising oxytocin in concentration of 10 mU/ml to 60 mU/ml, L-glutamic acid and lactobionic, where L-glutamic acid and lactobionic acid are each in concentration from 0.8 mg/ml to 2 mg/ml, wherein the pH is in the range of 3.8 to 5.5 and where the plastic bag is overwrapped.

The present disclosure provides a ready-to-administer formulation in a plastic bag, formulation comprising oxytocin in concentration of 10 mU/ml to 60 mU/ml, L-glutamic acid and lactobionic, where L-glutamic acid and lactobionic acid are each in concentration from 0.8 mg/ml to 2 mg/ml, wherein the pH is in the range of 3.8 to 5.5, and where the plastic bag is overwrapped and were the space between the plastic bag and the overwrap is not filled with nitrogen.

The present disclosure provides a ready-to-administer formulation in a plastic bag, formulation comprising oxytocin in concentration of 10 mU/ml to 60 mU/ml, L-glutamic acid and lactobionic, where L-glutamic acid and lactobionic acid are each in concentration from 0.8 mg/ml to 2 mg/ml, wherein the pH is in the range of 3.8 to 5.5, and where the plastic bag comprises EVOH barrier, plastic bag is overwrapped and were the space between the plastic bag and the overwrap is not filled with nitrogen.

The present disclosure provides a ready-to-administer formulation in a plastic bag, formulation comprising oxytocin in concentration of 10 mU/ml to 60 mU/ml, L-glutamic acid and lactobionic, wherein the pH is in the range of 3.8 to 5.5, and where the plastic bag comprises EVOH barrier.

The present disclosure provides a ready-to-administer formulation in a plastic bag, formulation comprising 10 mU/ml to 60 mU/ml oxytocin, L-glutamic acid and lactobionic, where L-glutamic acid and lactobionic acid are each in concentration from 0.1 mg/ml to 5 mg/ml, wherein the pH is in the range of 3.8 to 5.5, and where the plastic bag comprises EVOH barrier.

The present disclosure provides a ready-to-administer formulation in a plastic bag, formulation comprising oxytocin in concentration of 10 mU/ml to 60 mU/ml, L-glutamic acid and lactobionic, where L-glutamic acid and lactobionic acid are each in concentration from 0.8 mg/ml to 2 mg/ml, wherein the pH is in the range of 3.8 to 5.5, and where the plastic container bag EVOH barrier.

Any of the ready-to-administer formulations described above can have not more than 2% of dimer impurity when subjected to a temperature of 70° C. for 2 to 5 days.

Any of the ready-to-administer formulations described above can have not more than 3% of dimer impurity when subjected to a temperature of 70° C. for 2 to 5 days.

Any of the ready-to-administer formulations described above can have not more than 4% of dimer impurity when subjected to a temperature of 70° C. for 2 to 5 days.

Other objects, features and advantages will become apparent from the following detailed description and examples. It should be understood, however, that the detailed description and the examples, while indicating specific embodiments, are given by way of illustration only, and are not intended to limit the breadth or scope of the concepts in any manner.

Stabilizing effect of different factors in ready-to administer formulations were evaluated.

### GENERAL TECHNIQUES

All formulations presented in examples below were prepared by providing a liquid solution of oxytocin and at least one excipient. If necessary, pH was adjusted to achieve the desired pH range.

Prepared compositions were transferred into containers, such as vials, bags to achieve desirable amount of active component per container.

Formulations of oxytocin with 0.9% NaCl having the desired pH and stored in the different packaging are prepared and shown for comparative purposes.

### ANALYTICAL METHODS

After preparation, initial time point levels of active pharmaceutical ingredient and impurities were analyzed by ultra-high performance liquid chromatography (UHPLC) and afterwards containers were loaded into stability chambers at different storage conditions, 40° C. and 25° C.

In order to determine the stability of the active pharmaceutical ingredient in formulations according to the present disclosure, containers were taken from stability chambers at various time points, such as 14 days, 1 month, 2 months, 3 months, 4 months, 6 months etc., and analyzed by UHPLC.

The assay for active pharmaceutical ingredient was analyzed by separation by UHPLC and determination using fluorescence detector (FLD), a mass spectrometer (MS) with the addition of an internal standard and/or a diode array detector (DAD). Impurities were separated by UHPLC and determined with coupled MS or DAD. In all cases, a reverse-phase C18 column was used. The conditions for each method are listed below.

### Assay Procedure I (Fluorescence Detector)

Chromatographic System:
Detector: Fluorescence detector, excitation 228 nm, emission 310 nm
Column: 2.1-mm; 15-cm; 1.7 μm packing L1, Stationary phase: octadecylsilyl silica gel for chromatography R
Column temperature: 50° C.
Autosampler: 5° C.
Flow rate: 0.5 mL/min
Injection volume: 100 μL
Diluent: 5 g chlorobutanol, 5.0 mL acetic acid, 1.1 g sodium acetate and 4.7 g ethanol in 1 L of ultrapure water.
Solution A: 100 mM monobasic sodium phosphate, pH approximately 4.5
Solution B: water and acetonitrile (50:50)
Mobile phase: see table below

| Time (min) | Solution A (%) | Solution B (%) |
|---|---|---|
| 0 | 70 | 30 |
| 5 | 70 | 30 |
| 20 | 60 | 40 |
| 31.3 | 50 | 50 |
| 41 | 0 | 100 |
| 44 | 0 | 100 |
| 46.3 | 70 | 30 |
| 49.3 | 70 | 30 |

Sample solution preparation for formulation: directly injected into the system

Standard solution preparation: 0.06 IU/mL of Oxytocin Reference Standard in diluent

### Assay Procedure IIA (Mass Spectrometer)

Chromatographic System:
Detector: Electrospray ionization—triple quadrupole mass spectrometer. Gas temperature 300° C., gas flow 6 L/min, nebulizer 20 psi, sheath gas temperature 350° C., sheath gas flow 12 L/min, capillary 4000 V, operated in positive ion mode. Acquisition: multiple reaction monitoring. For quantitation of Oxytocin, precursor m/z 1007.1 was selected, while the monitored product m/z was 723. Internal standard (IS) Heavy Oxytocin was monitored at precursor m/z 1021.1, while the product m/z was 723 (or the most abundant product ion in the spectrum, if observed otherwise). In both cases dwell time, fragmentor voltage and collision energy were 200 ms, 238 V and 40 eV, respectively.
Column: 2.1-mm; 15-cm; 1.7 μm packing L1, Stationary phase: octadecylsilyl silica gel for chromatography R
Column temperature: 50° C.
Autosampler: 10° C.
Flow rate: 0.4 mL/min
Injection volume: 10 μL
Diluent preparation: 0.5 g chlorobutanol, 5.0 mL acetic acid, 1.1 g sodium acetate and 4.7 g ethanol in 1 L of ultrapure water.
IS preparation: Dissolve 25 nmol of Heavy Oxytocin in 2.0 mL of ultrapure water. Transfer 1.0 mL of obtained solution into a 20-mL volumetric flask and dilute to volume with ultrapure water.
Solution A: 10 mM ammonium formate, pH=4.5
Solution B: acetonitrile, MS grade
Mobile phase: see table below

| Time (min) | Solution A (%) | Solution B (%) |
|---|---|---|
| 0 | 85 | 15 |
| 8 | 79.4 | 20.6 |
| 9.5 | 10 | 90 |
| 11 | 10 | 90 |
| 12 | 85 | 15 |
| 14 | 85 | 15 |

Sample solution preparation for formulation: Add 50 μL of IS solution to 250 μL of sample solution, mix and inject into the system.

Standard solution preparation: 0.06 IU/mL of Oxytocin Reference Standard in diluent. Add 50 μL of IS solution to 250 μL of Oxytocin Reference Standard solution, mix and inject into the system.

### Assay Procedure IIB (Mass Spectrometer)

Chromatographic System:
Detector: Electrospray ionization—single quadrupole mass spectrometer. Gas temperature 300° C., gas flow 6 L/min, nebulizer 20 psi, sheath gas temperature 350° C., sheath gas flow 12 L/min, capillary 4000 V, nozzle voltage 0 V, operated in positive ion mode. For quantitation of Oxytocin, 1007.5 m/z was selected, while the IS Heavy Oxytocin was monitored at 1012.5 m/z.
Column: 2.1-mm; 5-cm; 1.7 μm packing L1, Stationary phase: octadecylsilyl silica gel for chromatography R
Column temperature: 40° C.
Flow rate: 0.4 mL/min
Injection volume: 20 μL
Diluent preparation: 0.5 g chlorobutanol, 5.0 mL acetic acid, 1.1 g sodium acetate and 4.7 g ethanol in 1 L of ultrapure water.
IS preparation: Dissolve 25 nmol of Heavy Oxytocin in 2.0 mL of ultrapure water. Transfer 1.0 mL of obtained solution into a 20-mL volumetric flask and dilute to volume with ultrapure water.
Solution A: 10 mM ammonium formate, pH=4.5
Solution B: acetonitrile, MS grade
Mobile phase: see table below

| Time (min) | Solution A (%) | Solution B (%) |
|---|---|---|
| 0 | 85 | 15 |
| 4 | 77 | 23 |
| 4.1 | 40 | 60 |
| 6 | 40 | 60 |
| 6.1 | 85 | 15 |
| 7.5 | 85 | 15 |

Sample solution preparation for formulation: Add 50 μL of IS solution to 250 μL of sample solution, mix and inject into the system.

Standard solution preparation: 0.06 IU/mL of Oxytocin Reference Standard in diluent. Add 50 μL of IS solution to 250 μL of Oxytocin Reference Standard solution, mix and inject into the system.

### Assay Procedure III (Diode Array Detector)

Chromatographic System:
Detector: Diode array detector at 205 nm, bandwidth 4 nm, no reference wavelength
Column: 2.1-mm; 15-cm; 1.7 μm packing L1, Stationary phase: octadecylsilyl silica gel for chromatography R
Column temperature: 40° C.
Autosampler: 5° C.
Flow rate: 0.4 mL/min
Injection volume: 40 μL
Diluent: 5 g chlorobutanol, 5.0 mL acetic acid, 1.1 g sodium acetate and 4.7 g ethanol in 1 L of ultrapure water.
Solution A: 10 mM monobasic sodium phosphate, pH=4.50
Solution B: acetonitrile
Mobile phase: see table below

| Time (min) | Solution A (%) | Solution (%) |
|---|---|---|
| 0.0 | 85.0 | 15.0 |
| 9.0 | 81.5 | 18.5 |
| 9.5 | 40.0 | 60.0 |
| 13.5 | 40.0 | 60.0 |
| 14.0 | 85.0 | 15.0 |

Sample solution preparation for formulation: directly injected into the system Standard solution preparation: 0.06 IU/mL of Oxytocin Reference Standard in diluent

### Impurities Procedure (Mass Spectrometer)

Chromatographic System:
Detector: Electrospray ionization—quadrupole-time-of-flight mass spectrometer. Capillary voltage 3000 V, sampling cone voltage 40 V, source temperature 120° C., desolvation temperature 350° C., desolvation gas flow 600 L/hr, operated in positive ion mode under resolution mode. Acquisition: scan from 100 to 1500 m/z at a frequency of 1 Hz.
Column: 2.1-mm; 10-cm; 1.7 μm packing L1, Stationary phase: octadecylsilyl silica gel for chromatography R
Column temperature: 50° C.
Autosampler: 10° C.
Flow rate: 0.4 mL/min
Injection volume: 10 μL
Solution A: 10 mM ammonium formate, pH=4.5
Solution B: acetonitrile, MS grade
Mobile phase: see table below

| Time (min) | Solution A (%) | Solution B (%) |
|---|---|---|
| 0 | 85 | 15 |
| 10 | 78 | 22 |
| 15 | 60 | 40 |
| 17 | 10 | 90 |
| 19 | 10 | 90 |
| 21 | 85 | 15 |
| 25 | 85 | 15 |

Sample solution preparation for formulation: Solid phase extraction.

### Calculation of the Assay of Active Compound Using Assay Procedure I (Fluorescence Detector) and III (Diode Array Detector)

The assay of the active compound is calculated using the following equation:

Assay (%)=(Amps/Ampstd)×(cstd/cs)×100%
where:
Amps=peak area of the Oxytocin peak in Sample solution
Ampstd=peak area of the Oxytocin peak in Standard solution
cs=concentration of Oxytocin in the Sample solution (IU/mL)
cstd=concentration of Oxytocin in the Standard solution (IU/mL)

### Calculation of the Assay of Active Compound Using Assay Procedure II (Mass Spectrometer)

The assay of the active compound is calculated using the following equation:

Assay (%)=[(AOs/AHs)/(AOstd/AHstd)]×cstd/cs×100%
where:
AOs=peak area of the Oxytocin peak in Sample solution
AHs=peak area of the IS peak in Sample solution
AOstd=peak area of the Oxytocin peak in Standard solution
AHstd=peak area of the IS peak in Standard solution
cs=concentration of Oxytocin in the Sample solution (IU/mL)
cstd=concentration of Oxytocin in the Standard solution (IU/mL)

### Calculation of the Assay Drop of the Main Peak

Assay(tp) (%)—Value of the assay of the main peak at time point (tp) different than initial, for example: 7 days, 1 month, 2 months etc. at different storage conditions such as 25° C., 40° C., analyzed by UHPLC
Assay(st) (%)—Value of the assay of the main peak at initial time point
ΔAssay—Calculated assay drop of the main compound: (%)

ΔAssay(%)=Assay(st) (%)−Assay(tp)(%)

Calculation of the Individual Impurity (Other than Higher-Order Structure Impurities)

The content of impurities monitored using the scan mode of the mass spectrometer, is given as area % of the total area.

### Calculation of the Higher-Order Structure (Dimer) Impurity

The amount of dimer(s) (expressed as %) at a certain timepoint is calculated as dimer concentration (in ng/mL) at a certain timepoint divided with Oxytocin concentration (in ng/mL) at start.

The amount of dimer(s) %=Dimer concentration at certain time point (ng/ml)/Oxytocin concentration at start (ng/ml)×100%
Sample dimer concentrations are calculated versus an external dimer standard using the following equation:

Dimer concentration (ng/mL)=(Amps/Ampstd)×(cstd/cs)
where:
Amps=peak area of the dimer peak in Sample solution
Ampstd=peak area of the dimer peak in Standard solution
cs=concentration of dimer in the Sample solution (ng/mL)
cstd=concentration of dimer in the Standard solution (ng/MI)

Above calculation may be used for determining other higher-order structure impurities when appropriately adjusted.

### Visible Particle Inspection

At the beginning of visual inspection at each stability time point, the bottom of the container is checked for precipitate formation, then visual particle inspection is checked by gently swirling and inverting containers, ensuring that no air bubbles are produced. Containers were inspected for approximately 15 sec with naked eye. Observation regarding visual inspection were noted for inspected samples.

List of abbreviations used:
HPBCD: 2-hydroxypropyl-beta-cyclodextrin
M: Month(s)
MonO: Monodeaminated Oxytocin
OTris: Oxytocin trisulfide
N/A: Not available

### EXAMPLES

In below Examples, oxytocin is added in the form of an acetate salt.

### Example 1

Ready-to-administer formulations of oxytocin were prepared by dissolving excipients and oxytocin separately in water or predefined standard diluent for parenteral use under predefined conditions. The contents were stirred using a magnetic stirrer. If necessary, pH was adjusted to a predefined pH. The solutions were mixed to ensure homogeneity, filtered through a 0.2 μm filter, transferred to a container and WFI or predefined diluent was added to make up to a predefined volume. The prepared formulations were then aliquoted in predefined packaging.

Formulations were then stored under controlled room temperature and stability was determined at time points such as 1 months, 2 months, 3 months, 6 months and further.

It is expected that the formulations of the disclosure are stable for 3 and 6 months under controlled room temperature and that this can be extrapolated to indicate that the formulations are stable or stabilized for up to about 12 months.
Table 1 shows chemical stability of ready-to-administer formulations comprising oxytocin and different excipients. Unless otherwise stated, formulations were stored in glass vials. Assay % was analyzed using UHPLC-FLD method.

TABLE 1
CHEMICAL STABILITY OF READY-TO-ADMINISTER
FORMULATIONS COMPRISING OXYTOCIN
AND DIFFERENT EXCIPIENTS

| | | | Time- | Assay | Δ Assay |
|---|---|---|---|---|---|
| Formulation | pH | Condition | point | % | % |
| Oxytocin 60 mIU/ml | 5.0 | 25° C. | Start | 99.0 | N/A |
| Sodium chloride 9.0 g/L | | | 3 M | 72.5 | 26.8 |
| | | 40° C. | 1 M | 45.0 | 54.6 |
| Oxytocin 60 mIU/ml | 3.8 | 25° C. | Start | 101.0 | N/A |
| L-Glutamic acid 1.6 g/L | | | 3 M | 100.5 | 0.5 |
| Sodium gluconate 1.5 g/L | | 40° C. | 1 M | 95.3 | 5.6 |
| Sodium chloride 9.0 g/L | | | | | |
| Oxytocin 60 mIU/ml | 4.1 | 25° C. | Start | 99.0 | N/A |
| L-Glutamic acid 1.6 g/L | | | 3 M | 99.2 | 0.2 |
| Sodium gluconate 0.35 g/L | | 40° C. | 1 M | 93.0 | 6.1 |
| Lactobionic acid 1.0 g/L | | | | | |
| Sodium chloride 9.0 g/L | | | | | |
| Oxytocin 60 mIU/ml | 3.8 | 25° C. | Start | 100.5 | N/A |
| Sodium gluconate 1.5 g/L | | | 3 M | 100.0 | 0.5 |
| Lactobionic acid 1.0 g/L | | 40° C. | 1 M | 92.8 | 7.6 |
| Sodium chloride 9.0 g/L | | | | | |

Table 2 shows the chemical stability of ready-to-administer formulations comprising oxytocin and different excipients. A formulation of oxytocin dissolved in 0.9% NaCl, with the pH adjusted to 4.0 was prepared for comparative purposes. Unless otherwise stated, formulations were stored in plastic bags comprising PE and EVOH and then overwrapped without using nitrogen blanketing or oxygen scavenger. Assay % was analyzed using UHPLC-DAD method.

TABLE 2
CHEMICAL STABILITY OF READY-TO-ADMINISTER FORMULATIONS
COMPRISING OXYTOCIN AND DIFFERENT EXCIPIENTS

| Formulation | pH | Condition | Timepoint | Assay % | Δ Assay % | MonO % | Otris % |
|---|---|---|---|---|---|---|---|
| Oxytocin 60 | 4.4 | 25° C. | start | 102.2 | N/A | <0.05 | 0.06 |
| mIU/ml; | | | 1 M | 101.8 | 0.3 | 0.10 | 0.09 |
| L-glutamic acid | | | 3 M | 101.5 | 0.7 | 0.26 | 0.14 |
| 0.883 g/L; | | 40° C. | 1 M | 98.3 | 3.8 | 0.46 | 0.18 |
| Lactobionic acid | | | | | | | |
| 0.95 g/L; | | | | | | | |
| NaCl 9.0 g/L | | | | | | | |
| Oxytocin 60 | 4.4 | 25° C. | start | 100.2 | N/A | <0.05 | 0.07 |
| mIU/ml; | | | 1 M | 100.0 | 0.2 | 0.10 | 0.38 |
| Sodium gluconate | | | 3 M | 98.8 | 1.3 | 0.22 | 0.58 |
| 1.308 g/L; | | 40° C. | 1 M | 93.7 | 6.5 | 0.49 | 0.9 |
| Lactobionic acid | | | | | | | |
| 0.5465 g/L; | | | | | | | |
| 2-picolinic acid | | | | | | | |
| 0.377 g/L; | | | | | | | |
| NaCl 9.0 g/L | | | | | | | |
| Oxytocin 60 | 4.0 | 25° C. | start | 103.3 | N/A | <0.05 | 0.07 |
| mIU/ml; | | | 1 M | 102.5 | 0.8 | 0.09 | 0.06 |
| Sodium gluconate | | | 3 M | 102.9 | 0.4 | 0.21 | 0.09 |
| 1.308 g/L; | | 40° C. | 1 M | 102.0 | 1.3 | 0.35 | 0.12 |
| NaCl 9.0 g/L | | | | | | | |
| Oxytocin 60 | 4.0 | 25° C. | start | 99.3 | N/A | <0.05 | 0.08 |
| mIU/ml; | | | 1 M | 97.8 | 1.5 | 0.18 | 0.08 |
| Sodium gluconate | | | 3 M | 96.5 | 2.8 | 0.44 | 0.11 |
| 1.308 g/L; | | 40° C. | 1 M | 101.0 | −1.7 | 0.74 | 0.13 |
| Lactobionic acid | | | | | | | |
| 0.95 g/l; | | | | | | | |
| NaCl 9.0 g/L | | | | | | | |
| Oxytocin 60 | 4.0 | 25° C. | start | 104.2 | N/A | <0.05 | <0.05 |
| mIU/ml; | | | 1 M | 103.8 | 0.3 | 0.11 | 0.09 |
| L-glutamic acid | | | 3 M | 102.7 | 1.5 | 0.27 | 0.15 |
| 0.883 g/L; | | 40° C. | 1 M | 102.5 | 1.7 | 0.45 | 0.18 |
| Sodium gluconate | | | | | | | |
| 0.883 g/L; | | | | | | | |
| NaCl 9.0 g/L | | | | | | | |
| Oxytocin 60 | 4.0 | 25° C. | start | 101.5 | N/A | <0.05 | <0.05 |
| mIU/ml; | | | 1 M | 101.3 | 0.2 | 0.20 | 0.10 |
| Adipic acid 0.877 | | | 3 M | 98.4 | 3.1 | 0.53 | 0.16 |
| g/L; | | 40° C. | 1 M | 101.7 | −0.2 | 0.77 | 0.17 |
| NaCl 9.0 g/L | | | | | | | |
| Oxytocin 60 | 3.5 | 25° C. | start | 102.3 | N/A | <0.05 | <0.05 |
| mIU/ml; | | | 1 M | 102.3 | 0.0 | 0.37 | 0.08 |
| Lactobionic acid | | | 3 M | 101.3 | 1.0 | 1.02 | 0.13 |
| 0.95 g/L; | | 40° C. | 1 M | 99.5 | 2.8 | 1.65 | 0.15 |
| NaCl 9.0 g/L | | | | | | | |
| Oxytocin 60 | 4.0 | 25° C. | start | 103.7 | N/A | 0.05 | 0.06 |
| mIU/ml; | | | 1 M | 103.3 | 0.3 | 0.21 | 0.12 |
| Lactobionic acid | | | 3 M | 101.5 | 2.2 | 0.53 | 0.17 |

| Formulation | pH | Condition | Container | Timepoint | Assay % | Δ Assay % | MonO | Otris |
|---|---|---|---|---|---|---|---|---|
| Oxytocin 60 | 4.4 | 25° C. | bag | start | 102.2 | N/A | N/A | 0.06 |
| mIU/ml; | | | | 6 M | 101.2 | 1.0 | 0.47 | 0.09 |
| L-glutamic acid | | | vial | start | 101.7 | N/A | N/A | 0.08 |
| 0.883 g/L; | | | | 6 M | 98.0 | 3.7 | 0.44 | 0.64 |
| Lactobionic acid | | | | | | | | |
| 0.95 g/L; | | | | | | | | |
| NaCl 9.0 g/L | | | | | | | | |
| Oxytocin 60 | 4.0 | 25° C. | bag | start | 103.3 | N/A | <0.05 | 0.07 |
| mIU/ml; Sodium | | | | 6 M | 99.9 | 3.4 | 0.53 | 0.16 |
| gluconate 1.308 | | | vial | start | 103.7 | N/A | <0.05 | <0.05 |
| g/L; | | | | 6 M | 96.2 | 7.5 | 0.49 | 0.69 |
| NaCl 9.0 g/L | | | | | | | | |
| Oxytocin 60 | 4.0 | 25° C. | bag | start | 104.2 | N/A | <0.05 | <0.05 |
| mIU/ml; | | | | 6 M | 102.2 | 2.0 | 0.66 | 0.24 |
| L-glutamic acid | | | vial | start | 103.0 | N/A | <0.05 | 0.05 |
| 0.883 g/L; | | | | 6 M | 97.2 | 5.8 | 0.59 | 1.53 |
| Sodium gluconate | | | | | | | | |
| 0.883 g/L; | | | | | | | | |
| NaCl 9.0 g/L | | | | | | | | |

| | | | | | | |
|---|---|---|---|---|---|---|
| 0.95 g/L; | 40° C. | 1 M | 100.8 | 2.8 | 0.86 | 0.17 |
| NaCl 9.0 g/L | | | | | | |

Table 3 shows the chemical stability of ready-to-administer formulations comprising oxytocin and different excipients in bags and glass vials. The plastic bags used in this example comprise polyethylene and EVOH. Bags were overwrapped without using nitrogen blanketing or oxygen scavenger. Assay % was analyzed using the UHPLC-DAD method while the impurities were analyzed by the UHPLC-MS method.
TABLE 3: Chemical stability of ready-to-administer formulations comprising oxytocin and different excipients stored in vials and bags.

### Example 2

Ready-to-administer formulations of oxytocin are prepared by dissolving excipients in water for injection or predefined standard diluent for parenteral use under predefined conditions until a clear solution (without visible particles) was obtained. Then oxytocin was added and the solution was mixed until oxytocin was dissolved. If necessary, pH was adjusted to a predefined pH value, and then WFI or appropriate diluent was added to make up to a predefined volume. The solution was mixed to ensure homogeneity, and afterwards filtered through a 0.2 μm filter, and aliquoted in predefined packaging.

1. A ready-to-administer formulation comprising oxytocin, gluconic acid or its pharmaceutically acceptable salt, wherein the pH of the formulation is in the range of 3.2 to 5.5.

2. The ready-to-administer formulation according to claim 1, where the gluconic acid salt is sodium gluconate.

3. The ready-to-administer formulation according to claim 1, where the concentration of oxytocin is from 5 mU/ml to 100 mU/ml.

4. The ready-to-administer formulation according to claim 3, where the concentration of oxytocin is from 10 mU/ml to 60 mU/ml.

5. The ready-to-administer formulation according to claim 1, where the concentration of gluconic acid or its pharmaceutically acceptable salt is from 0.5 mM to 200 mM.

6. The ready-to-administer formulation according to claim 1, where the concentration of gluconic acid or its pharmaceutically acceptable salt is from 0.5 mM to 200 mM.

7. The ready-to-administer formulation according to claim 1, further comprising glutamic acid or its pharmaceutically acceptable salt.

8. The ready-to-administer formulation according to claim 7, where the glutamic acid is L-glutamic acid.

9. The ready-to-administer formulation according to claim 1, further comprising lactobionic acid or its pharmaceutically acceptable salt.

10. The ready-to-administer formulation according to claim 1, wherein the formulation is stored in the plastic bag.

11. The ready-to-administer formulation according to claim 1, wherein the formulation is stable for 3 months under room temperature conditions, wherein stable means having no more than an 8% drop of oxytocin assay as determined by UHPLC.

12. A plastic bag comprising the ready-to-administer formulation according to claim 1.
